SEQUENCE LISTING (42 sequences)

SEQ ID NO: 1 - RNA, 23 nt, Artificial Sequence
  miR-29b mimic second-sense-passenger strand
  modified_base (1)..(1): O-methyl adenosine
  modified_base (2)..(2): O-methyl adenosine
  modified_base (3)..(3): O-methyl cytidine
  modified_base (5)..(5): O-methyl cytidine
  modified_base (6)..(6): O-methyl uridine
  modified_base (8)..(8): O-methyl uridine
  modified_base (9)..(9): O-methyl uridine
  modified_base (10)..(10): O-methyl uridine
  modified_base (12)..(12): O-methyl cytidine
  modified_base (16)..(16): O-methyl uridine
  modified_base (19)..(19): O-methyl uridine
  modified_base (20)..(20): O-methyl cytidine
  modified_base (21)..(21): O-methyl cytidine
  modified_base (22)..(22): O-methyl uridine
  misc_feature (23)..(23): modified with a cholesterol conjugate with a 6 carbon linker
aacacuguuuacaaaugguccua

SEQ ID NO: 2 - RNA, 25 nt, Artificial Sequence
  miR-29b mimic first-antisense-guide strand
  misc_feature (1)..(1): modified with a monophosphate moiety
  modified_base (1)..(1): fluoro uridine
  modified_base (4)..(4): fluoro cytidine
  modified_base (6)..(6): fluoro cytidine
  modified_base (7)..(7): fluoro cytidine
  modified_base (9)..(9): fluoro uridine
  modified_base (10)..(10): fluoro uridine
  modified_base (11)..(11): fluoro uridine
  modified_base (16)..(16): fluoro uridine
  modified_base (17)..(17): fluoro cytidine
  modified_base (20)..(20): fluoro uridine
  modified_base (22)..(22): fluoro uridine
  modified_base (23)..(23): fluoro uridine
  misc_feature (23)..(25): joined through phosphorothioate bonds
uagcaccauuugaaaucaguguuuu

SEQ ID NO: 3 - RNA, 24 nt, Artificial Sequence
  miR-29a mimic second-sense-passenger strand
  modified_base (1)..(1): O-methyl uridine
  modified_base (2)..(2): O-methyl adenosine
uaaccgauuucagauggugcuauu

SEQ ID NO: 4 - RNA, 22 nt, Artificial Sequence
  miR-29a mimic second-sense-passenger strand
  modified_base (1)..(1): O-methyl uridine
  modified_base (2)..(2): O-methyl adenosine
  modified_base (4)..(4): O-methyl cytidine
  modified_base (5)..(5): O-methyl cytidine
  modified_base (7)..(7): O-methyl uridine
  modified_base (8)..(8): O-methyl uridine
  modified_base (9)..(9): O-methyl uridine
  modified_base (11)..(11): O-methyl cytidine
  modified_base (15)..(15): O-methyl uridine
  modified_base (18)..(18): O-methyl uridine
  modified_base (19)..(19): O-methyl cytidine
  modified_base (20)..(20): O-methyl cytidine
  modified_base (21)..(21): O-methyl uridine
uaaccguuuacagaugguccua

SEQ ID NO: 5 - RNA, 22 nt, Artificial Sequence
  miR-29a mimic second-sense-passenger strand
  modified_base (1)..(1): O-methyl uridine
  modified_base (2)..(2): O-methyl adenosine
  modified_base (4)..(4): O-methyl cytidine
  modified_base (5)..(5): O-methyl cytidine
  modified_base (7)..(7): O-methyl uridine
  modified_base (8)..(8): O-methyl uridine
  modified_base (9)..(9): O-methyl uridine
  modified_base (11)..(11): O-methyl cytidine
  modified_base (15)..(15): O-methyl uridine
  modified_base (18)..(18): O-methyl uridine
  modified_base (19)..(19): O-methyl cytidine
  modified_base (20)..(20): O-methyl cytidine
  modified_base (21)..(21): O-methyl uridine
  misc_feature (22)..(22): modified with a cholesterol conjugate with a 6 carbon linker
uaaccguuuacagaugguccua

SEQ ID NO: 6 - RNA, 24 nt, Artificial Sequence
  miR-29a mimic first-antisense-guide strand
  misc_feature (1)..(1): modified with a monophosphate moiety
uagcaccaucugaaaucgguuauu

SEQ ID NO: 7 - RNA, 24 nt, Artificial Sequence
  miR-29a mimic first-antisense-guide strand
  misc_feature (1)..(1): modified with a monophosphate moiety
  modified_base (1)..(1): fluoro uridine
  modified_base (4)..(4): fluoro cytidine
  modified_base (6)..(6): fluoro cytidine
  modified_base (7)..(7): fluoro cytidine
  modified_base (9)..(9): fluoro uridine
  modified_base (10)..(10): fluoro cytidine
  modified_base (11)..(11): fluoro uridine
  modified_base (16)..(16): fluoro uridine
  modified_base (17)..(17): fluoro cytidine
  modified_base (20)..(20): fluoro uridine
  modified_base (21)..(21): fluoro uridine
  misc_feature (22)..(24): joined through phosphorothioate bonds
uagcaccaucugaaaucgguuauu

SEQ ID NO: 8 - RNA, 25 nt, Artificial Sequence
  miR-29b mimic second-sense-passenger strand
  modified_base (1)..(1): O-methyl adenosine
  modified_base (2)..(2): O-methyl adenosine
aacacugauuucaaauggugcuauu

SEQ ID NO: 9 - RNA, 23 nt, Artificial Sequence
  miR-29b mimic second-sense-passenger strand
  modified_base (1)..(1): O-methyl adenosine
  modified_base (2)..(2): O-methyl adenosine
  modified_base (3)..(3): O-methyl cytidine
  modified_base (5)..(5): O-methyl cytidine
  modified_base (6)..(6): O-methyl uridine
  modified_base (9)..(9): O-methyl uridine
  modified_base (10)..(10): O-methyl uridine
  modified_base (11)..(11): O-methyl uridine
  modified_base (12)..(12): O-methyl cytidine
  modified_base (16)..(16): O-methyl uridine
  modified_base (19)..(19): O-methyl uridine
  modified_base (21)..(21): O-methyl cytidine
  modified_base (22)..(22): O-methyl uridine
  misc_feature (23)..(23): modified with a cholesterol conjugate with a 6 carbon linker
aacacugauuucaaauggugcua

SEQ ID NO: 10 - RNA, 25 nt, Artificial Sequence
  miR-29b mimic second-sense-passenger strand
  modified_base (1)..(1): O-methyl adenosine
  modified_base (2)..(2): O-methyl adenosine
  misc_feature (23)..(25): joined through phosphorothioate bonds
  misc_feature (25)..(25): modified with a cholesterol conjugate with a 6 carbon linker
aacacugauuucaaauggugcuauu

SEQ ID NO: 11 - RNA, 24 nt, Artificial Sequence
  miR-29a mimic second-sense-passenger strand
  modified_base (1)..(1): O-methyl uridine
  modified_base (2)..(2): O-methyl adenosine
  misc_feature (22)..(24): joined through phosphorothioate bonds
  misc_feature (24)..(24): modified with a cholesterol conjugate with a 6 carbon linker
uaaccgauuucagauggugcuauu

SEQ ID NO: 12 - RNA, 24 nt, Artificial Sequence
  miR-29c mimic second-sense-passenger strand
  modified_base (1)..(1): O-methyl uridine
  modified_base (2)..(2): O-methyl adenosine
  misc_feature (22)..(24): joined through phosphorothioate bonds
  misc_feature (24)..(24): modified with a cholesterol conjugate with a 6 carbon linker
uaaccgauuucaaauggugcuauu

SEQ ID NO: 13 - RNA, 23 nt, Artificial Sequence
  miR-29b mimic second-sense-passenger strand
  modified_base (1)..(1): O-methyl adenosine
  modified_base (2)..(2): O-methyl adenosine
  modified_base (3)..(3): O-methyl cytidine
  modified_base (5)..(5): O-methyl cytidine
  modified_base (6)..(6): O-methyl uridine
  modified_base (8)..(8): O-methyl uridine
  modified_base (9)..(9): O-methyl uridine
  modified_base (10)..(10): O-methyl uridine
  modified_base (12)..(12): O-methyl cytidine
  modified_base (16)..(16): O-methyl uridine
  modified_base (19)..(19): O-methyl uridine
  modified_base (20)..(20): O-methyl cytidine
  modified_base (21)..(21): O-methyl cytidine
  modified_base (22)..(22): O-methyl uridine
aacacuguuuacgggugguccua

SEQ ID NO: 14 - RNA, 23 nt, Artificial Sequence
  miR-29b mimic second-sense-passenger strand
  modified_base (1)..(1): O-methyl adenosine
  modified_base (2)..(2): O-methyl adenosine
  modified_base (3)..(3): O-methyl cytidine
  modified_base (5)..(5): O-methyl cytidine
  modified_base (6)..(6): O-methyl uridine
  modified_base (8)..(8): O-methyl uridine
  modified_base (9)..(9): O-methyl uridine
  modified_base (10)..(10): O-methyl uridine
  modified_base (12)..(12): O-methyl cytidine
  modified_base (16)..(16): O-methyl uridine
  modified_base (19)..(19): O-methyl uridine
  modified_base (20)..(20): O-methyl cytidine
  modified_base (21)..(21): O-methyl cytidine
  modified_base (22)..(22): O-methyl uridine
  misc_feature (23)..(23): modified with a cholesterol conjugate with a 6 carbon linker
aacacuguuuacgggugguccua

SEQ ID NO: 15 - DNA, 25 bp, Artificial Sequence
  miR-29b mimic second-sense-passenger strand
  modified_base (1)..(1): O-methyl adenosine
  modified_base (2)..(2): O-methyl adenosine
  modified_base (3)..(3): O-methyl cytidine
  modified_base (4)..(4): ribonucleotide adenosine
  modified_base (5)..(5): O-methyl cytidine
  modified_base (6)..(6): O-methyl uridine
  modified_base (7)..(7): ribonucleotide guanosine
  modified_base (8)..(8): O-methyl uridine
  modified_base (9)..(9): O-methyl uridine
  modified_base (10)..(10): O-methyl uridine
  modified_base (11)..(11): ribonucleotide adenosine
  modified_base (12)..(12): O-methyl cytidine
  modified_base (13)..(13): ribonucleotide adenosine
  modified_base (14)..(14): ribonucleotide adenosine
  modified_base (15)..(15): ribonucleotide adenosine
  modified_base (16)..(16): O-methyl uridine
  modified_base (17)..(17): ribonucleotide guanosine
  modified_base (18)..(18): ribonucleotide guanosine
  modified_base (19)..(19): O-methyl uridine
  modified_base (20)..(20): O-methyl cytidine
  modified_base (21)..(21): O-methyl cytidine
  modified_base (22)..(22): O-methyl uridine
  modified_base (23)..(23): ribonucleotide adenosine
  misc_feature (25)..(25): modified with a cholesterol conjugate with a 6 carbon linker
aacacuguuuacaaaugguccuatt

SEQ ID NO: 16 - DNA, 25 bp, Artificial Sequence
  miR-29b mimic second-sense-passenger strand
  misc_feature (1)..(1): modified with a cholesterol conjugate with a 6 carbon linker
  modified_base (3)..(3): O-methyl adenosine
  modified_base (4)..(4): O-methyl adenosine
  modified_base (5)..(5): O-methyl cytidine
  modified_base (6)..(6): ribonucleotide adenosine
  modified_base (7)..(7): O-methyl cytidine
  modified_base (8)..(8): O-methyl uridine
  modified_base (9)..(9): ribonucleotide guanosine
  modified_base (10)..(10): O-methyl uridine
  modified_base (11)..(11): O-methyl uridine
  modified_base (12)..(12): O-methyl uridine
  modified_base (13)..(13): ribonucleotide adenosine
  modified_base (14)..(14): O-methyl cytidine
  modified_base (15)..(15): ribonucleotide adenosine
  modified_base (16)..(16): ribonucleotide adenosine
  modified_base (17)..(17): ribonucleotide adenosine
  modified_base (18)..(18): O-methyl uridine
  modified_base (19)..(19): ribonucleotide guanosine
  modified_base (20)..(20): ribonucleotide guanosine
  modified_base (21)..(21): O-methyl uridine
  modified_base (22)..(22): O-methyl cytidine
  modified_base (23)..(23): O-methyl cytidine
  modified_base (24)..(24): O-methyl uridine
  modified_base (25)..(25): ribonucleotide adenosine
ttaacacuguuuacaaaugguccua

SEQ ID NO: 17 - RNA, 23 nt, Artificial Sequence
  miR-29b mimic second-sense-passenger strand
  modified_base (1)..(1): O-methyl adenosine
  modified_base (2)..(2): O-methyl adenosine
  modified_base (3)..(3): O-methyl cytidine
  modified_base (5)..(5): O-methyl cytidine
  modified_base (6)..(6): O-methyl uridine
  modified_base (8)..(8): O-methyl uridine
  modified_base (9)..(9): O-methyl uridine
  modified_base (10)..(10): O-methyl uridine
  modified_base (12)..(12): O-methyl cytidine
  modified_base (16)..(16): O-methyl uridine
  modified_base (19)..(19): O-methyl uridine
  modified_base (20)..(20): O-methyl cytidine
  modified_base (21)..(21): O-methyl cytidine
  modified_base (22)..(22): O-methyl uridine
  misc_feature (23)..(23): modified with a cholesterol conjugate with a 9 carbon linker
aacacuguuuacaaaugguccua

SEQ ID NO: 18 - RNA, 25 nt, Artificial Sequence
  miR-29b mimic first-antisense-guide strand
  misc_feature (1)..(1): modified with a monophosphate moiety
uagcaccauuugaaaucaguguuuu

SEQ ID NO: 19 - RNA, 25 nt, Artificial Sequence
  miR-29b mimic first-antisense-guide strand
  modified_base (1)..(1): fluoro uridine
  modified_base (4)..(4): fluoro cytidine
  modified_base (6)..(6): fluoro cytidine
  modified_base (7)..(7): fluoro cytidine
  modified_base (9)..(9): fluoro uridine
  modified_base (10)..(10): fluoro uridine
  modified_base (11)..(11): fluoro uridine
  modified_base (16)..(16): fluoro uridine
  modified_base (17)..(17): fluoro cytidine
  modified_base (20)..(20): fluoro uridine
  modified_base (22)..(22): fluoro uridine
  modified_base (23)..(23): fluoro uridine
  misc_feature (23)..(25): joined through phosphorothioate bonds
uagcaccauuugaaaucaguguuuu

SEQ ID NO: 20 - RNA, 25 nt, Artificial Sequence
  miR-29b mimic first-antisense-guide strand
  misc_feature (1)..(1): modified with a monophosphate moiety
  modified_base (1)..(1): fluoro uridine
  modified_base (4)..(4): fluoro cytidine
  modified_base (6)..(6): fluoro cytidine
  modified_base (7)..(7): fluoro cytidine
  modified_base (9)..(9): fluoro cytidine
  modified_base (10)..(10): fluoro cytidine
  modified_base (11)..(11): fluoro cytidine
  modified_base (16)..(16): fluoro uridine
  modified_base (17)..(17): fluoro cytidine
  modified_base (20)..(20): fluoro uridine
  modified_base (22)..(22): fluoro uridine
  modified_base (23)..(23): fluoro uridine
  misc_feature (23)..(25): joined through phosphorothioate bonds
uagcaccacccgaaaucaguguuuu

SEQ ID NO: 21 - RNA, 25 nt, Artificial Sequence
  miR-29b mimic first-antisense-guide strand
uagcaccauuugaaaucaguguuuu

SEQ ID NO: 22 - RNA, 24 nt, Artificial Sequence
  miR-29c mimic second-sense-passenger strand
  modified_base (1)..(1): O-methyl uridine
  modified_base (2)..(2): O-methyl adenosine
uaaccgauuucaaauggugcuauu

SEQ ID NO: 23 - RNA, 22 nt, Artificial Sequence
  miR-29c mimic second-sense-passenger strand
  modified_base (1)..(1): O-methyl uridine
  modified_base (2)..(2): O-methyl adenosine
  modified_base (4)..(4): O-methyl cytidine
  modified_base (5)..(5): O-methyl cytidine
  modified_base (7)..(7): O-methyl uridine
  modified_base (8)..(8): O-methyl uridine
  modified_base (9)..(9): O-methyl uridine
  modified_base (11)..(11): O-methyl cytidine
  modified_base (15)..(15): O-methyl uridine
  modified_base (18)..(18): O-methyl uridine
  modified_base (19)..(19): O-methyl cytidine
  modified_base (20)..(20): O-methyl cytidine
  modified_base (21)..(21): O-methyl uridine
uaaccguuuacaaaugguccua

SEQ ID NO: 24 - RNA, 22 nt, Artificial Sequence
  miR-29c mimic second-sense-passenger strand
  modified_base (1)..(1): O-methyl uridine
  modified_base (2)..(2): O-methyl adenosine
  modified_base (4)..(4): O-methyl cytidine
  modified_base (5)..(5): O-methyl cytidine
  modified_base (7)..(7): O-methyl uridine
  modified_base (8)..(8): O-methyl uridine
  modified_base (9)..(9): O-methyl uridine
  modified_base (11)..(11): O-methyl cytidine
  modified_base (15)..(15): O-methyl uridine
  modified_base (18)..(18): O-methyl uridine
  modified_base (19)..(19): O-methyl cytidine
  modified_base (20)..(20): O-methyl cytidine
  modified_base (21)..(21): O-methyl uridine
  misc_feature (22)..(22): modified with a cholesterol conjugate with a 6 carbon linker
uaaccguuuacaaaugguccua

SEQ ID NO: 25 - RNA, 24 nt, Artificial Sequence
  miR-29c mimic first-antisense-guide strand
  misc_feature (1)..(1): modified with a monophosphate moiety
uagcaccauuugaaaucgguuauu

SEQ ID NO: 26 - RNA, 24 nt, Artificial Sequence
  miR-29c mimic first-antisense-guide strand
  misc_feature (1)..(1): modified with a monophosphate moiety
  modified_base (1)..(1): fluoro uridine
  modified_base (4)..(4): fluoro cytidine
  modified_base (6)..(6): fluoro cytidine
  modified_base (7)..(7): fluoro cytidine
  modified_base (9)..(9): fluoro uridine
  modified_base (10)..(10): fluoro uridine
  modified_base (11)..(11): fluoro uridine
  modified_base (16)..(16): fluoro uridine
  modified_base (17)..(17): fluoro cytidine
  modified_base (20)..(20): fluoro uridine
  modified_base (21)..(21): fluoro uridine
  misc_feature (22)..(24): joined through phosphorothioate bonds
uagcaccauuugaaaucgguuauu

SEQ ID NO: 27 - RNA, 24 nt, Artificial Sequence
  miR-29a mimic first-antisense-guide strand
  modified_base (1)..(1): fluoro uridine
  modified_base (4)..(4): fluoro cytidine
  modified_base (6)..(6): fluoro cytidine
  modified_base (7)..(7): fluoro cytidine
  modified_base (9)..(9): fluoro uridine
  modified_base (10)..(10): fluoro cytidine
  modified_base (11)..(11): fluoro uridine
  modified_base (16)..(16): fluoro uridine
  modified_base (17)..(17): fluoro cytidine
  modified_base (20)..(20): fluoro uridine
  modified_base (21)..(21): fluoro uridine
  misc_feature (22)..(24): joined through phosphorothioate bonds
uagcaccaucugaaaucgguuauu

SEQ ID NO: 28 - RNA, 23 nt, Artificial Sequence
  miR-29b mimic second-sense-passenger strand
  modified_base (2)..(2): O-methyl adenosine
  modified_base (4)..(4): O-methyl adenosine
  modified_base (6)..(6): O-methyl uridine
  modified_base (8)..(8): O-methyl adenosine
  modified_base (10)..(10): O-methyl uridine
  modified_base (12)..(12): O-methyl cytidine
  modified_base (14)..(14): O-methyl adenosine
  modified_base (16)..(16): O-methyl uridine
  modified_base (18)..(18): O-methyl guanosine
  modified_base (20)..(20): O-methyl guanosine
  modified_base (22)..(22): O-methyl uridine
  misc_feature (23)..(23): modified with a cholesterol conjugate with a 6 carbon linker
aacacugauuucaaauggugcua

SEQ ID NO: 29 - RNA, 25 nt, Artificial Sequence
  miR-29b mimic second-sense-passenger strand
  modified_base (2)..(2): O-methyl adenosine
  modified_base (4)..(4): O-methyl adenosine
  modified_base (6)..(6): O-methyl uridine
  modified_base (8)..(8): O-methyl adenosine
  modified_base (10)..(10): O-methyl uridine
  modified_base (12)..(12): O-methyl cytidine
  modified_base (14)..(14): O-methyl adenosine
  modified_base (16)..(16): O-methyl uridine
  modified_base (18)..(18): O-methyl guanosine
  modified_base (20)..(20): O-methyl guanosine
  modified_base (22)..(22): O-methyl uridine
  misc_feature (23)..(25): joined through phosphorothioate bonds
  misc_feature (25)..(25): modified with a cholesterol conjugate with a 6 carbon linker
aacacugauuucaaauggugcuauu

SEQ ID NO: 30 - RNA, 23 nt, Artificial Sequence
  miR-29b mimic second-sense-passenger strand
  modified_base (1)..(1): O-methyl adenosine
  modified_base (2)..(2): O-methyl adenosine
  modified_base (3)..(3): O-methyl cytidine
  modified_base (5)..(5): O-methyl cytidine
  modified_base (6)..(6): O-methyl uridine
  modified_base (8)..(8): O-methyl uridine
  modified_base (9)..(9): O-methyl uridine
  modified_base (10)..(10): O-methyl uridine
  modified_base (12)..(12): O-methyl cytidine
  modified_base (16)..(16): O-methyl uridine
  modified_base (19)..(19): O-methyl uridine
  modified_base (20)..(20): O-methyl cytidine
  modified_base (21)..(21): O-methyl cytidine
  modified_base (22)..(22): O-methyl uridine
  modified_base (23)..(23): modified with a cholesterol conjugate with a tetraethylene glycol moiety
aacacuguuuacaaaugguccua

SEQ ID NO: 31 - RNA, 23 nt, Artificial Sequence
  miR-29b mimic first-antisense-guide strand
  modified_base (1)..(1): O-methyl uridine
  modified_base (3)..(3): O-methyl guanosine
  modified_base (5)..(5): O-methyl adenosine
  modified_base (7)..(7): O-methyl cytidine
  modified_base (9)..(9): O-methyl uridine
  modified_base (11)..(11): O-methyl uridine
  modified_base (13)..(13): O-methyl adenosine
  modified_base (15)..(15): O-methyl adenosine
  modified_base (17)..(17): O-methyl cytidine
  modified_base (19)..(19): O-methyl guanosine
  modified_base (21)..(21): O-methyl guanosine
  modified_base (23)..(23): O-methyl uridine
uagcaccauuugaaaucaguguu

SEQ ID NO: 32 - RNA, 25 nt, Artificial Sequence
  miR-29b mimic first-antisense-guide strand
  modified_base (1)..(1): O-methyl uridine
  modified_base (3)..(3): O-methyl guanosine
  modified_base (5)..(5): O-methyl adenosine
  modified_base (7)..(7): O-methyl cytidine
  modified_base (9)..(9): O-methyl uridine
  modified_base (11)..(11): O-methyl uridine
  modified_base (13)..(13): O-methyl adenosine
  modified_base (15)..(15): O-methyl adenosine
  modified_base (17)..(17): O-methyl cytidine
  modified_base (19)..(19): O-methyl guanosine
  modified_base (21)..(21): O-methyl guanosine
  modified_base (23)..(23): O-methyl uridine
  misc_feature (23)..(25): joined through phosphorothioate bonds
uagcaccauuugaaaucaguguuuu

SEQ ID NO: 33 - RNA, 25 nt, Artificial Sequence
  miR-29b mimic first-antisense-guide strand
  modified_base (1)..(1): O-methyl uridine
  modified_base (4)..(4): O-methyl cytidine
  modified_base (6)..(6): O-methyl cytidine
  modified_base (7)..(7): O-methyl cytidine
  modified_base (9)..(9): O-methyl uridine
  modified_base (10)..(10): O-methyl uridine
  modified_base (11)..(11): O-methyl uridine
  modified_base (16)..(16): O-methyl uridine
  modified_base (17)..(17): O-methyl cytidine
  modified_base (20)..(20): O-methyl uridine
  modified_base (22)..(22): O-methyl uridine
  modified_base (23)..(23): O-methyl uridine
  misc_feature (23)..(25): joined through phosphorothioate bonds
uagcaccauuugaaaucaguguuuu

SEQ ID NO: 34 - RNA, 23 nt, Artificial Sequence
  miR-29b mimic first-antisense-guide strand
  modified_base (1)..(1): fluoro uridine
  modified_base (4)..(4): fluoro cytidine
  modified_base (6)..(6): fluoro cytidine
  modified_base (7)..(7): fluoro cytidine
  modified_base (9)..(9): fluoro uridine
  modified_base (10)..(10): fluoro uridine
  modified_base (11)..(11): fluoro uridine
  modified_base (16)..(16): fluoro uridine
  modified_base (17)..(17): fluoro cytidine
  modified_base (20)..(20): fluoro uridine
  modified_base (22)..(22): fluoro uridine
  modified_base (23)..(23): fluoro uridine
uagcaccauuugaaaucaguguu

SEQ ID NO: 35 - RNA, 24 nt, Artificial Sequence
  miR-29c mimic first-antisense-guide strand
  modified_base (1)..(1): fluoro uridine
  modified_base (4)..(4): fluoro cytidine
  modified_base (6)..(6): fluoro cytidine
  modified_base (7)..(7): fluoro cytidine
  modified_base (9)..(9): fluoro uridine
  modified_base (10)..(10): fluoro uridine
  modified_base (11)..(11): fluoro uridine
  modified_base (16)..(16): fluoro uridine
  modified_base (17)..(17): fluoro cytidine
  modified_base (20)..(20): fluoro uridine
  modified_base (21)..(21): fluoro uridine
  misc_feature (22)..(24): joined through phosphorothioate bonds
uagcaccauuugaaaucgguuauu

SEQ ID NO: 36 - skipped

SEQ ID NO: 37 - RNA, 22 nt, Artificial Sequence
  miR-29a mimic second-sense-passenger strand
  modified_base (1)..(1): O-methyl uridine
  modified_base (2)..(2): O-methyl adenosine
uaaccgauuucagauggugcua

SEQ ID NO: 38 - RNA, 24 nt, Artificial Sequence
  miR-29a mimic first-antisense-guide strand
uagcaccaucugaaaucgguuauu

SEQ ID NO: 39 - RNA, 23 nt, Artificial Sequence
  miR-29b mimic second-sense-passenger strand
  modified_base (1)..(1): O-methyl adenosine
  modified_base (2)..(2): O-methyl adenosine
aacacugauuucaaauggugcua

SEQ ID NO: 40 - RNA, 25 nt, Artificial Sequence
  miR-29b mimic first-antisense-guide strand
uagcaccauuugaaaucaguguuuu

SEQ ID NO: 41 - RNA, 22 nt, Artificial Sequence
  miR-29c mimic second-sense-passenger strand
  modified_base (1)..(1): O-methyl uridine
  modified_base (2)..(2): O-methyl adenosine
uaaccgauuucaaauggugcua

SEQ ID NO: 42 - RNA, 24 nt, Artificial Sequence
  miR-29c mimic first-antisense-guide strand
uagcaccauuugaaaucgguuauu

(19) United States
(12) Patent Application Publication
(10) Pub. No.: US 2020/0376019 A1
(43) Pub. Date: Dec. 3, 2020

(54) miR29 MIMICS FOR THE TREATMENT OF OCULAR FIBROSIS

(71) Applicant: MIRAGEN THERAPEUTICS, INC., Boulder, CO (US)

(72) Inventors: Corrie Lynn GALLANT-BEHM, Boulder, CO (US); Aimee JACKSON, Boulder, CO (US); Stephanie PROPP, Boulder, CO (US); Paul D. RUBIN, Boulder, CO (US)

(21) Appl. No.: 16/768,514

(22) PCT Filed: Nov. 30, 2018
(86) PCT No.: PCT/US2018/063458

§ 371 Date: May 29, 2020

Related U.S. Application Data
(60) Provisional application No. 62/593,198, filed on Nov. 30, 2017.
(60) Provisional application No. 62/694,936, filed on Jul. 6, 2018.

(51) Int. Cl. A61K 31/713; C12N 15/113

(52) U.S. Cl. CPC A61K 31/713; A61K 9/0019; C12N 15/113

(57) ABSTRACT

Provided herein are double-stranded, chemically-modified oligonucleotide mimetics of miR-29 for use in treating ocular diseases or disorders associated with ocular fibrosis, and ocular fibrotic conditions.

### CROSS-REFERENCE TO RELATED APPLICATIONS

This application claims the benefit of U.S. Provisional Patent Application No. 62/694,936, filed on Jul. 6, 2018, and U.S. Provisional Patent Application No. 62/593,198, filed on Nov. 30, 2017, both of which are hereby incorporated by reference in their entirety.

### BACKGROUND

MicroRNAs (miRNAs) have several advantages as therapeutic intervention points in that they are small and comprise a known sequence. Since a single miRNA can regulate numerous target mRNAs within biological pathways, modulation of a miRNA may allow for influencing an entire gene network and modifying complex disease phenotypes (van Rooij & Olson, 2012).

Homeostasis of the eye depends on the presence of normal vasculature and an extracellular matrix. If such homeostasis is disturbed by, for example, infection, inflammation, or metabolic disease, visual function becomes impaired. The end result of these conditions is often fibrosis. Disruption of the highly ordered tissue architecture in the eye caused by fibrosis can lead to mechanical disruption of the visual axis and/or biological malfunctioning (Friedlander M., Journal of Clinical Investigation. 2007; 117(3):576-586).

There is an unmet medical need for interventions that can effectively treat ocular fibrotic conditions. Provided herein are methods and compositions that address this need.

### SUMMARY

Provided herein are double-stranded RNA miR-29 mimetic compounds (mimics) for the treatment of ocular fibrotic conditions.

In a first aspect, the disclosure provides a method of treating ocular fibrosis in a subject comprising administering to the subject a therapeutically effective amount of a miR-29 mimetic compound, wherein the miR-29 mimetic compound comprises a first strand of 23 to 26 ribonucleotides comprising a mature miR-29a, miR-29b, or miR-29c sequence; and a second strand of 22 to 23 ribonucleotides comprising a sequence that is substantially complementary to the first strand and having at least one modified nucleotide, wherein the first strand has a 3′ nucleotide overhang relative to the second strand. In some embodiments, the ocular fibrosis comprises retinal fibrosis, corneal fibrosis, conjunctival fibrosis, or fibrosis of the trabecular meshwork.

In some embodiments, the ocular fibrosis results from an ocular disease or disorder selected from the group consisting of diabetic retinopathy, bacterial infection, viral infection, fungal infection, amoeba infection, ocular trauma, chemical corneal burn, thermal corneal burn, pterygium, glaucoma, surgical trauma, Fuch's Endothelial Corneal Dystrophy (FECD), and anterior proliferative vitreoretinopathy (anterior PVR).

In a second aspect, the disclosure provides a method of treating an ocular disease or disorder in a subject comprising administering to the subject a therapeutically effective amount of a miR-29 mimetic compound, wherein the miR-29 mimetic compound comprises a first strand of 23 to 26 ribonucleotides comprising a mature miR-29a, miR-29b, or miR-29c sequence; and a second strand of 22 to 23 ribonucleotides comprising a sequence that is substantially complementary to the first strand and having at least one modified nucleotide, wherein the first strand has a 3′ nucleotide overhang relative to the second strand. In some embodiments, the ocular disease or disorder is selected from the group consisting of diabetic retinopathy, bacterial infection, viral infection, fungal infection, amoeba infection, ocular trauma, chemical corneal burn, thermal corneal burn, pterygium, glaucoma, surgical trauma, Fuch's Endothelial Corneal Dystrophy (FECD), and anterior proliferative vitreoretinopathy (anterior PVR).

In some embodiments, the ocular disease or disorder comprises an ocular fibrosis selected from the group consisting of comprises retinal fibrosis, corneal fibrosis, conjunctival fibrosis, or fibrosis of the trabecular meshwork

In some embodiments, the miR-29 mimetic compound is administered via iontophoresis, subconjunctival injection, injection into the pterygium, injection at site of a trabecular bleb, sub-tendon injection, an intravitreal injection, intracamerally, or topically.

In some embodiments, the ocular fibrosis comprises proliferative vitreoretinopathy.

In some embodiments, the ocular fibrosis comprises retinal fibrosis associated with diabetic retinopathy.

In some embodiments, the ocular fibrosis comprises retinal fibrosis associated with anti-VEGF treatment.

In some embodiments, the ocular fibrosis comprises corneal hazing or corneal scarring.

In some embodiments, the ocular fibrosis results from a corneal burn.

In some embodiments, the ocular fibrosis results from pterygium.

In some embodiments, the ocular fibrosis results from Fuch's Endothelial Corneal Dystrophy (FECD).

In some embodiments, the ocular fibrosis results from glaucoma.

In some embodiments, the ocular fibrosis comprises a trabecular bleb.

In some embodiments, the miR-29 mimetic compound is administered topically into the cornea.

In some embodiments, the miR-29 mimetic compound is administered topically into the cornea in a drop formulation.

In some embodiments, the miR-29 mimetic compound is administered topically into the cornea once daily, twice daily, three times daily, or four times daily.

In some embodiments, the miR-29 mimetic compound is administered topically periodically for up to one week, two weeks, three weeks, four weeks, or 28 days.

In some embodiments, the miR-29 mimetic compound is administered in a drop formulation.

In some embodiments, the miR-29 mimetic compound is administered via intravitreal injection once weekly.

In some embodiments, the miR-29 mimetic compound is administered via intravitreal injection periodically for one month.

In some embodiments, the miR-29 mimetic compound is presented in Table 2.

In some embodiments, the expression of COL1A1, COL1A2, COL3A1, COL4A3, COL5A2, COL11A1, FN1, MMP2, CTGF, TGFB2, and/or TGFB3 is reduced.

In some embodiments, the first strand comprises a sequence selected from SEQ ID NO: 2, 18-21, or 31-34 and the second strand comprises a sequence selected from 1, 8-10, 13-17, or 28-30.

In some embodiments, the miR-29 mimetic compound comprises a first strand of about 23 to about 26 ribonucleotides comprising a mature miR-29b sequence, wherein the first strand comprises a sequence selected from SEQ ID NO: 2, 18-21, and 31-34; and a second strand of about 22 to about 25 ribonucleotides comprising a sequence that is substantially complementary to the first strand and having at least one modified nucleotide, wherein the first strand has a 3′ nucleotide overhang relative to the second strand.

In some embodiments, the miR-29 mimetic compound comprises a first strand of about 23 to about 26 ribonucleotides comprising a mature miR-29b sequence; and a second strand of about 22 to about 25 ribonucleotides comprising a sequence that is substantially complementary to the first strand and having at least one modified nucleotide, wherein the first strand has a 3′ nucleotide overhang relative to the second strand, and wherein the second strand comprises a sequence selected from SEQ ID NO: 1, 8-10, 13-17, and 28-30.

In some embodiments, the first strand comprises the sequence of SEQ ID NO: 19 and the second strand comprises the sequence of SEQ ID NO: 1.

In some embodiments, the first strand comprises the sequence of SEQ ID NO: 19 and the second strand comprises the sequence of SEQ ID NO: 15.

In some embodiments, the first strand comprises a mature miR-29a sequence.

In some embodiments, the miR-29 mimetic compound is presented in Table 1.

In some embodiments, the first strand comprises a sequence of SEQ ID NO: 6, 7 or 27 and the second strand comprises a sequence selected from SEQ ID NO: 3-5 or 11.

In some embodiments, the first strand comprises a mature miR-29c sequence.

In some embodiments, the miR-29 mimetic compound is presented in Table 3.

In some embodiments, the first strand comprises a sequence of SEQ ID NO: 25, 26, or 35 and the second strand comprises a sequence selected from SEQ ID NO: 22-24 or 12.

In some embodiments, a therapeutically effective amount of the miR-29 mimetic compound is comprised in a pharmaceutical composition.

In some embodiments, the subject is a human.

In some embodiments, the subject suffers from diabetes.

In some embodiments, the subject suffers from diabetic retinopathy.

In some embodiments, the subject suffers from age-related macular degeneration (AMD).

In some embodiments, the subject suffers from wet age-related macular degeneration (wet AMD).

In some embodiments, the subject is receiving an anti-VEGF therapy.

### BRIEF DESCRIPTION OF THE DRAWINGS

FIG. 1A shows a corneal burn created by administration of NaOH and FIG. 1B shows the uptake of an exemplary miR-29b mimic of the disclosure into the eye. FIG. 1C shows DAPI nuclear stain.

FIG. 2A and FIG. 2B show the biodistribution and pharmacokinetics (PK) of an exemplary miR-29b mimic of the disclosure in the eye.

FIG. 3A, FIG. 3B, FIG. 3C, and FIG. 3D show that an exemplary miR-29b mimic of the disclosure decreases target gene expression in vivo after corneal burn, across time points.

FIG. 4A and FIG. 4B shows the effect of miR-29b mimic administration on the histopathology of corneas harvested between days 4 and 14 after corneal injury.

FIG. 5A and FIG. 5B shows the effect of miR-29b mimic administration on the expression of alpha-smooth muscle actin (α-SMA) following corneal injury.

FIG. 6A and FIG. 6B shows the effect of miR-29b mimic administration on corneal scarring and hazing.

FIG. 7A, FIG. 7B, FIG. 7C, and FIG. 7D show the uptake of an exemplary miR-29b mimic of the disclosure into the eye.

FIG. 8A and FIG. 8B shows the biodistribution and pharmacokinetics (PK) of an exemplary miR-29b mimic of the disclosure in the eye.

FIG. 9A shows that an exemplary miR-29b mimic of the disclosure decreases target gene expression in vivo in proliferative vitreoretinopathy (PVR). FIG. 9B shows that an exemplary miR-29b mimic of the disclosure decreases target gene expression in vivo in diabetic retinopathy.

FIG. 10A, FIG. 10B, and FIG. 10C show the effect of an exemplary miR-29b mimic on target gene expression in vitro in induced pluripotent derived retinal pigment epithelial (iPS-RPE) and in primary human retinal pigment epithelial (hRPE) cells.

### DETAILED DESCRIPTION

Provided herein are double-stranded RNA miR-29 mimetic compounds (mimics) for the treatment of ocular fibrotic conditions, for example retinal fibrosis, corneal fibrosis, conjunctival fibrosis, or fibrosis of the trabecular meshwork.

MicroRNA Mimetic Compounds

A microRNA mimetic compound according to the disclosure comprises a first strand and a second strand, wherein the first strand comprises a mature miR-29a, miR-29b, or miR-29c sequence and the second strand comprises a sequence that is substantially complementary to the first strand and has at least one modified nucleotide. Throughout the disclosure, the term “microRNA mimetic compound” may be used interchangeably with the terms “promiR-29,” “miR-29 agonist,” “microRNA agonist,” “microRNA mimic,” “miRNA mimic,” or “miR-29 mimic;” the term “first strand” may be used interchangeably with the terms “antisense strand” or “guide strand”; and the term “second strand” may be used interchangeably with the term “sense strand” or “passenger strand”.

In some embodiments, the first strand of the microRNA mimetic compound comprises from about 23 to about 26 nucleotides comprising a sequence of mature miR-29a, miR-29b, or miR-29c and the second strand comprises from about 22 to about 23 nucleotides comprising a sequence that is partially, substantially, or fully complementary to the first strand. In various embodiments, the first strand may comprise about 23, 24, 25, or 26 nucleotides and the second strand may comprise about 22 or 23 nucleotides.

The nucleotides that form the first and the second strand of the microRNA mimetic compounds may comprise ribonucleotides, deoxyribonucleotides, modified nucleotides, and combinations thereof. In certain embodiments, the first strand and the second strand of the microRNA mimetic compound comprise ribonucleotides and/or modified ribonucleotides. The term “modified nucleotide” means a nucleotide where the nucleobase and/or the sugar moiety is modified relative to unmodified nucleotides.

In certain embodiments, the microRNA mimetic compounds have a first strand or an antisense strand, whose sequence is identical to all or part of a mature miR-29a, miR-29b, or miR-29c sequence, and a second strand or a sense strand whose sequence is about 70% to about 100% complementary to the sequence of the first strand. In some embodiments, the first strand of the miRNA mimetic compound is at least about 75, 80, 85, 90, 95, or 100% identical, including all integers there between, to the entire sequence of a mature, naturally occurring miR-29a, miR-29b, or miR-29c sequence. In certain embodiments, the first strand is about or is at least about 90, 91, 92, 93, 94, 95, 96, 97, 98, 99, or 100% identical to the sequence of a mature, naturally-occurring miRNA, such as the mouse, human, or rat miR-29a, miR-29b, or miR-29c sequence. Alternatively, the first strand may comprise 20, 21, 22, or 23 nucleotide positions in common with a mature, naturally-occurring miRNA as compared by sequence alignment algorithms and methods well known in the art.

It is understood that the sequence of the first strand is considered to be identical to the sequence of a mature miR-29a, miR-29b, or miR-29c even if the first strand includes a modified nucleotide instead of a naturally-occurring nucleotide. For example, if a mature, naturally-occurring miRNA sequence comprises a cytidine nucleotide at a specific position, the first strand of the mimetic compound may comprise a modified cytidine nucleotide, such as 2′-fluoro-cytidine, at the corresponding position or if a mature, naturally-occurring miRNA sequence comprises a uridine nucleotide at a specific position, the miRNA region of the first strand of the mimetic compound may comprise a modified uridine nucleotide, such as 2′-fluoro-uridine, 2′-O-methyl-uridine, 5-fluorouracil, or 4-thiouracil at the corresponding position. Thus, as long as the modified nucleotide has the same base-pairing capability as the nucleotide present in the mature, naturally-occurring miRNA sequence, the sequence of the first strand is considered to be identical to the mature, naturally-occurring miRNA sequence. In some embodiments, the first strand may include a modification of the 5′-terminal residue. For example, the first strand may have a 5′-terminal monophosphate. In some other embodiments, the first strand does not contain a 5′-terminal monophosphate.

In some embodiments, the second strand of the microRNA mimic is partially complementary to the sequence of the first strand. For example, the sequence of the second strand is at least about 70%, 75%, 80%, 85%, 90%, 95%, 96%, 97%, 98%, or 99%, inclusive of all values therebetween, complementary to the sequence of the first strand. In some other embodiments, the second strand is substantially complementary to the sequence of the first strand. For example, the second strand is at least about 85%, 86%, 87%, 88%, 89%, 90%, 91%, 92%, 93%, 94%, 95%, 96%, 97%, 98%, or 99%, inclusive of all values therebetween, complementary to the sequence of the first strand. In yet some other embodiments, the sequence of the second strand may be fully complementary to the first strand. In certain embodiments, about 19, 20, 21, 22, or 23 nucleotides of the complementary region of the second strand may be complementary to the first strand.

It is understood that the sequence of the second strand is considered to be complementary to the first strand even if the second strand includes a modified nucleotide instead of a naturally-occurring nucleotide. For example, if the first strand sequence comprises a guanosine nucleotide at a specific position, the second strand may comprise a modified cytidine nucleotide, such as 2′-O-methyl-cytidine, at the corresponding position.

In some embodiments, the second strand comprises about 1, 2, 3, 4, 5, or 6 mismatches relative to the first strand. That is, up to 1, 2, 3, 4, 5, or 6 nucleotides between the first strand and the second strand may not be complementary. In some embodiments, the mismatches are not consecutive and are distributed throughout the second strand. In another embodiment, the mismatches are consecutive and may create a bulge. In some embodiments, the second strand contains 3 mismatches relative to the first strand. In certain embodiments, the second strand of a miR-29a mimic or a miR-29c mimic contains mismatches at positions 4, 13, and/or 16 from the 3′ end (of the second strand) relative to the first strand. In some embodiments, the second strand of a miR-29b mimic contains mismatches at positions 4, 13, and/or 16 from the 3′ end (of the second strand) relative to the first strand. In another embodiment, the second strand of a miR-29b mimic contains mismatches at positions 4, 9, 10, 11, 13 and/or 16 from the 3′ end (of the second strand) relative to the first strand.

In some embodiments, the first and/or the second strand of the mimetic compound may comprise an overhang on the 5′ or 3′ end of the strands. In certain embodiments, the first strand comprises a 3′ overhang, i.e., a single-stranded region that extends beyond the duplex region, relative to the second strand. The 3′ overhang of the first strand may range from about one nucleotide to about four nucleotides. In certain embodiments, the 3′ overhang of the first strand may comprise 1 or 2 nucleotides. In some embodiments, the nucleotides comprising the 3′ overhang in the first strand are linked by phosphorothioate linkages. The nucleotides comprising the 3′ overhang in the first strand may include ribonucleotides, deoxyribonucleotides, modified nucleotides, or combinations thereof. In certain embodiments, the 3′ overhang in the first strand comprises two ribonucleotides. In some embodiments, the 3′ overhang of the first strand comprises two uridine nucleotides linked through a phosphorothioate linkage. In some embodiments, the first strand may not contain an overhang.

In some embodiments, the nucleotides in the second/sense strand of a miR-29 mimic of the disclosure is linked by phosphodiester linkages and the nucleotides in the first/antisense strand are linked by phosphodiester linkages except for the last three nucleotides at the 3′ end which are linked to each other via phosphorothioate linkages.

In various embodiments, the miR-29 mimic of the present disclosure comprise modified nucleotides. For instance, in some embodiments, the first strand of the mimic comprises one or more 2′-fluoro nucleotides. In another embodiment, the first strand may not include any modified nucleotide. In some embodiments, the second strand comprises one or more 2′-O-methyl modified nucleotides.

In various embodiments, a miR-29 mimic according to the present disclosure comprise first and second strands listed in the Tables below. Descriptions of the modifications are presented in Table 4. These miR-29 mimics have previously been described in International Publication WO/2016/040373; accordingly this application incorporates by reference International Publication WO/2016/040373 by Montgomery et al., published Mar. 17, 2016 herein in its entirety.

TABLE 1
miR-29a mimics

| | |
|---|---|
| | SEQ |
| | ID |
| Modified Sequence | NO. |

Second/sense/passenger strands

| | |
|---|---|
| 5′-mU.mA.rA.rC.rC.rG.rA.rU.rU.rU.rC.rA.rG.rA.rU. | 3 |
| rG.rG.rU.rG.rC.rU.rA.rU.rU-3′ | |
| 5′-mU.mA.rA.mC.mC.rG.mU.mU.mU.rA.mC.rA.rG.rA.mU. | 4 |
| rG.rG.mU.mC.mC.mU.rA-3′ | |
| 5′-mU.mA.rA.mC.mC.rG.mU.mU.mU.rA.mC.rA.rG.rA.mU. | 5 |
| rG.rG.mU.mC.mC.mU.rA.chol6-3′ | |
| 5′-mU.mA.rA.rC.rC.rG.rA.rU.rU.rU.rC.rA.rG.rA.rU. | 11 |
| rG.rG.rU.rG.rC.rU.rAs.rUs.rUs.chol6-3′ | |
| 5′-mU.mA.rA.rC.rC.rG.rA.rU.rU.rU.rC.rA.rG.rA.rU. | 37 |
| rG.rG.rU.rG.rC.rU.rA-3′ | |

First/antisense/guide strands

| | |
|---|---|
| 5′-p.rU.rA.rG.rC.rA.rC.rC.rA.rU.rC.rU.rG.rA.rA. | 6 |
| rA.rU.rC.rG.rG.rU.rU.rA.rU.rU-3′ | |
| 5′-p.fU.rA.rG.fC.rA.fC.fC.rA.fU.fC.fU.rG.rA.rA. | 7 |
| rA.fU.fC.rG.rG.fU.fU.rAs.rUs.rU-3′ | |
| 5′-fU.rA.rG.fC.rA.fC.fC.rA.fU.fC.fU.rG.rA.rA.rA. | 27 |
| fU.fC.rG.rG.fU.fU.rAs.rUs.rU-3′ | |
| 5′-rU.rA.rG.rC.rA.rC.rC.rA.rU.rC.rU.rG.rA.rA.rA. | 38 |
| rU.rC.rG.rG.rU.rU.rA.rU.rU-3′ | |

TABLE 2
miR-29b mimics

| | |
|---|---|
| | SEQ |
| | ID |
| Modified Sequence | NO. |

Second/sense/passenger strands

| | |
|---|---|
| 5′-mA.mA.rC.rA.rC.rU.rG.rA.rU.rU.rU.rC.rA.rA. | 8 |
| rA.rU.rG.rG.rU.rG.rC.rU.rA.rU.rU-3′ | |
| 5′-mA.mA.mC.rA.mC.mU.rG.rA.mU.mU.mU.mC.rA.rA. | 9 |
| rA.mU.rG.rG.mU.rG.mC.mU.rA.chol6-3′ | |
| 5′-mA.mA.rC.rA.rC.rU.rG.rA.rU.rU.rU.rC.rA.rA. | 10 |
| rA.rU.rG.rG.rU.rG.rC.rU.rAs.rUs.rUs.chol6-3′ | |
| 5′-mA.mA.mC.rA.mC.mU.rG.mU.mU.mU.rA.mC.rG.rG. | 13 |
| rG.mU.rG.rG.mU.mC.mC.mU.rA-3′ | |
| 5′-mA.mA.mC.rA.mC.mU.rG.mU.mU.mU.rA.mC.rG.rG. | 14 |
| rG.mU.rG.rG.mU.mC.mC.mU.rA.chol6-3′ | |
| 5′-mA.mA.mC.rA.mC.mU.rG.mU.mU.mU.rA.mC.rA.rA. | 1 |
| rA.mU.rG.rG.mU.mC.mC.mU.rA.chol6-3′ | |
| 5′-mA.mA.mC.rA.mC.mU.rG.mU.mU.mU.rA.mC.rA.rA. | 15 |
| rA.mU.rG.rG.mU.mC.mC.mU.rA.dT.dT.chol6-3′ | |
| 5′-C6Chol.dT.dT.mA.mA.mC.rA.mC.mU.rG.mU.mU. | 16 |
| mU.rA.mC.rA.rA.rA.mU.rG.rG.mU.mC.mC.mU.rA-3′ | |
| 5′-mA.mA.mC.rA.mC.mU.rG.mU.mU.mU.rA.mC.rA.rA. | 17 |
| rA.mU.rG.rG.mU.mC.mC.mU.rA.chol9-3′ | |
| 5′-rA.mA.rC.mA.rC.mU.rG.mA.rU.mU.rU.mC.rA.mA. | 28 |
| rA.mU.rG.mG.rU.mG.rC.mU.rA.chol6-3′ | |
| 5′-rA.mA.rC.mA.rC.mU.rG.mA.rU.mU.rU.mC.rA.mA. | 29 |
| rA.mU.rG.mG.rU.mG.rC.mU.rAs.rUs.rU.chol6-3′ | |
| 5′-mA.mA.mC.rA.mC.mU.rG.mU.mU.mU.rA.mC.rA.rA. | 30 |
| rA.mU.rG.rG.mU.mC.mC.mU.rA.cholTEG-3′ | |
| 5′-mA.mA.rC.rA.rC.rU.rG.rA.rU.rU.rU.rC.rA.rA. | 39 |
| rA.rU.rG.rG.rU.rG.rC.rU.rA-3′ | |

First/antisense/guide strands

| | |
|---|---|
| 5′-p.rU.rA.rG.rC.rA.rC.rC.rA.rU.rU.rU.rG.rA. | 18 |
| rA.rA.rU.rC.rA.rG.rU.rG.rU.rU.rU.rU-3′ | |
| 5′-p.fU.rA.rG.fC.rA.fC.fC.rA.fU.fU.fU.rG.rA. | 2 |
| rA.rA.fU.fC.rA.rG.fU.rG.fU.fUs.rUs.rU-3′ | |
| 5′-fU.rA.rG.fC.rA.fC.fC.rA.fU.fU.fU.rG.rA.rA. | 19 |
| rA.fU.fC.rA.rG.fU.rG.fU.fUs.rUs.rU-3′ | |
| 5′-p.fU.rA.rG.fC.rA.fC.fC.rA.fC.fC.fC.rG.rA. | 20 |
| rA.rA.fU.fC.rA.rG.fU.rG.fU.fUs.rUs.rU-3′ | |
| 5′-rU.rA.rG.rC.rA.rC.rC.rA.rU.rU.rU.rG.rA.rA. | 21 |
| rA.rU.rC.rA.rG.rU.rG.rU.rU.rU.rU-3′ | |
| 5′-mU.rA.mG.rC.mA.rC.mC.rA.mU.rU.mU.rG.mA.rA. | 31 |
| mA.rU.mC.rA.mG.rU.mG.rU.mU-3′ | |
| 5′ mU.rA.mG.rC.mA.rC.mC.rA.mU.rU.mU.rG.mA. | 32 |
| rA.mA.rU.mC.rA.mG.rU.mG.rU.mUs.rUs.rU-3′ | |
| 5′-mU.rA.rG.mC.rA.mC.mC.rA.mU.mU.mU.rG.rA.rA. | 33 |
| rA.mU.mC.rA.rG.mU.rG.mU.mUs.rUs.rU-3′ | |
| 5′-fU.rA.rG.fC.rA.fC.fC.rA.fU.fU.fU.rG.rA.rA. | 34 |
| rA.fU.fC.rA.rG.fU.rG.fU.fU-3′ | |
| 5′-rU.rA.rG.rC.rA.rC.rC.rA.rU.rU.rU.rG.rA.rA. | 40 |
| rA.rU.rC.rA.rG.rU.rG.rU.rU.rU.rU-3′ | |

TABLE 3
miR-29c mimics

| | |
|---|---|
| | SEQ |
| | ID |
| Modified Sequence | NO. |

Second/sense/passenger strands

| | |
|---|---|
| 5′-mU.mA.rA.rC.rC.rG.rA.rU.rU.rU.rC.rA.rA.rA. | 22 |
| rU.rG.rG.rU.rG.rC.rU.rA.rU.rU-3′ | |
| 5′-mU.mA.rA.mC.mC.rG.mU.mU.mU.rA.mC.rA.rA.rA. | 23 |
| mU.rG.rG.mU.mC.mC.mU.rA-3′ | |
| 5′-mU.mA.rA.mC.mC.rG.mU.mU.mU.rA.mC.rA.rA.rA. | 24 |
| mU.rG.rG.mU.mC.mC.mU.rA.chol6-3′ | |
| 5′-mU.mA.rA.rC.rC.rG.rA.rU.rU.rU.rC.rA.rA.rA. | 12 |
| rU.rG.rG.rU.rG.rC.rU.rAs.rUs.rUs.chol6-3′ | |
| 5′-mU.mA.rA.rC.rC.rG.rA.rU.rU.rU.rC.rA.rA.rA. | 41 |
| rU.rG.rG.rU.rG.rC.rU.rA-3′ | |

First/antisense/guide strands

| | |
|---|---|
| 5′-p.rU.rA.rG.rC.rA.rC.rC.rA.rU.rU.rU.rG.rA. | 25 |
| rA.rA.rU.rC.rG.rG.rU.rU.rA.rU.rU-3′ | |
| 5′-p.fU.rA.rG.fC.rA.fC.fC.rA.fU.fU.fU.rG.rA. | 26 |
| rA.rA.fU.fC.rG.rG.fU.fU.rAs.rUs.rU-3′ | |
| 5′-fU.rA.rG.fC.rA.fC.fC.rA.fU.fU.fU.rG.rA.rA. | 35 |
| rA.fU.fC.rG.rG.fU.fU.rAs.rUs.rU-3′ | |
| 5′-rU.rA.rG.rC.rA.rC.rC.rA.rU.rU.rU.rG.rA.rA. | 42 |
| rA.rU.rC.rG.rG.rU.rU.rA.rU.rU-3′ | |

TABLE 4
Abbreviations

| Nucleotide unit | | Nucleotide unit | |
|---|---|---|---|
| or modification | Abbreviation | or modification | Abbreviation |
| ribo A | rA | ribo A P═S | rAs |
| ribo G | rG | ribo G P═S | rGs |
| ribo C | rC | ribo C P═S | rCs |
| ribo U | rU | ribo U P═S | rUs |
| O-methyl A | mA | O-methyl A P═S | mAs |
| O-methyl G | mG | O-methyl G P═S | mGs |
| O-methyl C | mC | O-methyl C P═S | mCs |
| O-methyl U | mU | O-methyl U P═S | mUs |
| fluoro C | fC | fluoro C P═S | fCs |
| fluoro U | fU | fluoro U P═S | fUs |
| deoxy A | dA | deoxy A P═S | dAs |
| deoxy G | dG | deoxy G P═S | dGs |
| deoxy C | dC | deoxy C P═S | dCs |
| deoxy T | dT | deoxy T P═S | dTs |
| monophosphate | p | | |
| Cholesterol | Chol6/ | | |
| conjugate | C6 chol | | |
| with a 6 carbon | | | |
| linker | | | |
| Cholesterol | Chol9 | | |
| conjugate | | | |
| with a 9 carbon | | | |
| linker | | | |

In certain embodiments, a miR-29a mimic comprises a first strand comprising SEQ ID NO: 27 and a second strand comprising SEQ ID NO: 5. In other embodiments, a miR-29a mimic comprises a first strand comprising SEQ ID NO: 7 and a second strand comprising SEQ ID NO: 5.

In some embodiments, a miR-29b mimic comprises a first strand comprising SEQ ID NO: 19 and a second strand comprising SEQ ID NO: 1. In some other embodiments, a miR-29b mimic comprises a first strand comprising SEQ ID NO: 2 and a second strand comprising SEQ ID NO: 1. In yet some other embodiments, a miR-29b mimic comprises a first strand comprising SEQ ID NO: 19 and a second strand comprising SEQ ID NO: 15. In yet some other embodiments, a miR-29b mimic comprises a first strand comprising SEQ ID NO: 33 and a second strand comprising SEQ ID NO: 1. In yet some other embodiments, a miR-29b mimic comprises a first strand comprising SEQ ID NO: 34 and a second strand comprising SEQ ID NO: 1. In yet some other embodiments, a miR-29b mimic comprises a first strand comprising SEQ ID NO: 19 and a second strand comprising SEQ ID NO: 30.

In certain embodiments, a miR-29c mimic comprises a first strand comprising SEQ ID NO: 35 and a second strand comprising SEQ ID NO: 24. In other embodiments, a miR-29c mimic comprises a first strand comprising SEQ ID NO: 26 and a second strand comprising SEQ ID NO: 24.

In certain embodiments, the miR-29 mimetic compound comprises:

(a) a first strand of 23 to 26 ribonucleotides comprising a mature miR-29a, miR-29b, or miR-29c sequence; and

(b) a second strand of 22 to 23 ribonucleotides comprising a sequence that is substantially complementary to the first strand and having at least one modified nucleotide, wherein the first strand has a 3′ nucleotide overhang relative to the second strand.

In certain embodiments, the miR-29 mimetic compound comprises:

(c) a first strand of about 23 to about 26 ribonucleotides comprising a mature miR-29b sequence wherein the first strand comprises a sequence selected from SEQ ID NO: 2, 18-21, and 31-34; and

(d) a second strand of about 22 to about 25 ribonucleotides comprising a sequence that is substantially complementary to the first strand and having at least one modified nucleotide, wherein the first strand has a 3′ nucleotide overhang relative to the second strand.

In certain embodiments, the miR-29 mimetic compound comprises:

(a) a first strand of about 23 to about 26 ribonucleotides comprising a mature miR-29b sequence; and

(b) a second strand of about 22 to about 25 ribonucleotides comprising a sequence that is substantially complementary to the first strand and having at least one modified nucleotide, wherein the first strand has a 3′ nucleotide overhang relative to the second strand, and wherein the second strand comprises a sequence selected from SEQ ID NO: 1, 8-10, 13-17, and 28-30.

The modified nucleotides that may be used in the microRNA mimetic compounds of the disclosure can include nucleotides with a base modification or substitution. The natural or unmodified bases in RNA are the purine bases adenine (A) and guanine (G), and the pyrimidine bases cytosine (C) and uracil (U) (DNA has thymine (T)). In contrast, modified bases, also referred to as heterocyclic base moieties, include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (including 5-bromo, 5-trifluoromethyl and other 5-substituted uracils and cytosines), 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

In some embodiments, the microRNA mimetic compounds can have nucleotides with modified sugar moieties. Representative modified sugars include carbocyclic or acyclic sugars, sugars having substituent groups at one or more of their 2′, 3′ or 4′ positions and sugars having substituents in place of one or more hydrogen atoms of the sugar. In certain embodiments, the sugar is modified by having a substituent group at the 2′ position. In additional embodiments, the sugar is modified by having a substituent group at the 3′ position. In other embodiments, the sugar is modified by having a substituent group at the 4′ position. It is also contemplated that a sugar may have a modification at more than one of those positions, or that an RNA molecule may have one or more nucleotides with a sugar modification at one position and also one or more nucleotides with a sugar modification at a different position.

Sugar modifications contemplated in the miRNA mimetic compounds include, but are not limited to, a substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl.

In some embodiments, miRNA mimetic compounds have a sugar substituent group selected from the following: C₁ to C₁₀ lower alkyl, substituted lower alkyl, alkenyl, alkynyl, alkaryl, aralkyl, O-alkaryl or O-aralkyl, SH, SCH₃, Cl, Br, CN, OCN, CF₃, OCF₃, SOCH₃, SO₂CH₃, ONO₂, NO₂, N₃, NH₂, heterocycloalkyl, heterocycloalkaryl, aminoalkylamino, or similar substituents. In some embodiments, the modification includes 2′-methoxyethoxy (2′-O-CH₂CH₂OCH₃, which is also known as 2′-O-(2-methoxyethyl) or 2′-MOE), that is, an alkoxyalkoxy group. Another modification includes 2′-dimethylaminooxyethoxy, that is, a O(CH₂)₂₀N(CH₃)₂ group, also known as 2′-DMAOE and 2′-dimethylaminoethoxyethoxy (also known in the art as 2′-O-dimethyl-amino-ethoxy-ethyl or 2′-DMAEOE), that is, 2′-O—CH₂-O—CH₂-N(CH₃)₂.

Sugar substituent groups on the 2′ position (2′-) may be in the arabino (up) position or ribo (down) position. One 2′-arabino modification is 2′-F. Other similar modifications may also be made at other positions on the sugar moiety, particularly the 3′ position of the sugar on the 3′ terminal nucleoside or in 2′-5′ linked oligonucleotides and the 5′ position of 5′ terminal nucleotide.

In certain embodiments, the sugar modification is a 2′-O-alkyl (e.g. 2′-O-methyl, 2′-O-methoxyethyl), 2′-halo (e.g., 2′-fluoro, 2′-chloro, 2′-bromo), and 4′ thio modifications. For instance, in some embodiments, the first strand of the miR-29a, miR-29b, or miR-29c mimetic compound comprises one or more 2′ fluoro nucleotides. In another embodiment, the first strand of the mimetic compounds has no modified nucleotides. In yet another embodiment, the second strand of miR-29a, miR-29b, or miR-29c mimetic compound comprises one or more 2′-O-methyl modified nucleotides.

The first and the second strand of microRNA mimetic compounds of the disclosure can also include backbone modifications, such as one or more phosphorothioate, morpholino, or phosphonocarboxylate linkages (see, for example, U.S. Pat. Nos. 6,693,187 and 7,067,641, which are herein incorporated by reference in their entireties). For example, in some embodiments, the nucleotides comprising the 3′ overhang in the first strand are linked by phosphorothioate linkages.

In some embodiments, the microRNA mimetic compounds are conjugated to a carrier molecule such as a steroid (cholesterol), a vitamin, a fatty acid, a carbohydrate or glycoside, a peptide, or other small molecule ligand to facilitate in vivo delivery and stability. Preferably, the carrier molecule is attached to the second strand of the microRNA mimetic compound at its 3′ or 5′ end through a linker or a spacer group. In various embodiments, the carrier molecule is cholesterol, a cholesterol derivative, cholic acid or a cholic acid derivative. The use of carrier molecules disclosed in U.S. Pat. No. 7,202,227, which is incorporated by reference herein in its entirety, is also envisioned. In certain embodiments, the carrier molecule is cholesterol and it is attached to the 3′ or 5′ end of the second strand through at least a six carbon linker. In some embodiments, the carrier molecule is attached to the 3′ end of the second strand through a six or nine carbon linker. In some embodiments, the linker is a cleavable linker. In various embodiments, the linker comprises a substantially linear hydrocarbon moiety. The hydrocarbon moiety may comprise from about 3 to about 15 carbon atoms and may be conjugated to cholesterol through a relatively non-polar group such as an ether or a thioether linkage. In certain embodiments, the hydrocarbon linker/spacer comprises an optionally substituted C2 to C15 saturated or unsaturated hydrocarbon chain (e.g. alkylene or alkenylene). A variety of linker/spacer groups described in U.S. Pre-grant Publication No. 2012/0128761, which is incorporated by reference herein in its entirety, can be used in the present disclosure.

Methods of Use

In various embodiments, the present disclosure provides methods of treating, ameliorating, or preventing ocular fibrotic conditions in a subject in need thereof comprising administering to the subject a therapeutically effective amount of at least one of a miR-29a, miR-29b, and/or miR-29c mimic described herein. Use of miR-29 agonists in treating certain non-ocular fibrotic conditions is described in U.S. Pat. No. 8,440,636, which is hereby incorporated by reference in its entirety herein.

Ocular fibrotic conditions that may be treated using a miR-29 mimic of the disclosure include, but are not limited to retinal fibrosis, corneal fibrosis, conjunctival fibrosis, or fibrosis of the trabecular meshwork. In some embodiments, the ocular fibrosis comprises proliferative vitreoretinopathy. In some embodiments, the ocular fibrosis comprises retinal fibrosis associated with diabetic retinopathy. In some embodiments, the ocular fibrosis comprises retinal fibrosis associated with anti-VEGF treatment. In some embodiments, the ocular fibrosis comprises corneal hazing or corneal scarring, for example after photorefractive keratectomy (PRK) or trauma. In some embodiments, the ocular fibrosis results from PRK. In some embodiments, the ocular fibrosis results from trauma. In some embodiments, the ocular fibrosis results from infection. In some embodiments, the ocular fibrosis results from bacterial infection, viral infection, fungal infection, or amoeba infection. In some embodiments, the ocular fibrosis results from corneal transplant. In some embodiments, the ocular fibrosis comprises a corneal burn. In some embodiments, the corneal burn comprises a thermal corneal burn. In some embodiments, the corneal burn comprises a chemical corneal burn. In some embodiments, the ocular fibrosis comprises pterygium. In some embodiments, the ocular fibrosis comprises glaucoma. In some embodiments, the ocular fibrosis is fibrosis of a surgically created bleb that can lead to bleb failure (preventing fibrosis at the site of a surgically created trabecular bleb to prevent it from sealing shut) and leading to a recurrence of glaucoma (referred to herein as a trabecular bleb). In some embodiments, the ocular fibrosis comprises Fuch's Endothelial Corneal Dystrophy (FECD). In some embodiments, the ocular fibrosis comprises anterior proliferative vitreoretinopathy (anterior PVR).

In some embodiments, the disclosure provides a method of treating an ocular disease or disorder in a subject comprising administering to the subject a therapeutically effective amount of a miR-29 mimetic compound, wherein the miR-29 mimetic compound comprises a first strand of 23 to 26 ribonucleotides comprising a mature miR-29a, miR-29b, or miR-29c sequence; and a second strand of 22 to 23 ribonucleotides comprising a sequence that is substantially complementary to the first strand and having at least one modified nucleotide, wherein the first strand has a 3′ nucleotide overhang relative to the second strand, wherein the ocular disease or disorder is selected from the group consisting of diabetic retinopathy, bacterial infection, viral infection, fungal infection, amoeba infection, ocular trauma, chemical corneal burn, thermal corneal burn, pterygium, glaucoma, surgical trauma, Fuch's Endothelial Corneal Dystrophy (FECD), and anterior proliferative vitreoretinopathy (anterior PVR). In some embodiments, the ocular disease or disorder comprises an ocular fibrosis selected from the group consisting of comprises retinal fibrosis, corneal fibrosis, conjunctival fibrosis, or fibrosis of the trabecular meshwork.

In some embodiments, administration of a miR-29 mimic of the present disclosure reduces the expression or activity one or more extracellular matrix genes in cells of the subject. In another embodiment, administration of a miR-29 mimic of the present disclosure reduces the expression or activity one or more collagen synthesis genes in cells of the subject. In yet another embodiment, administration of a miR-29 mimic up-regulates the expression or activity one or more genes involved in the skin development, cornea development, epidermis development, ectoderm development, myofibroblast activation and cellular homeostasis. Cells of the subject where the expression or activity of various genes is regulated by a miR-29 mimic of the disclosure include fibroblasts, keratocytes, epidermal, epithelial and endothelial cells. In some embodiments, administration of a miR-29 mimic reduces the expression of COL1A1, COL1A2, COL3A1, COL4A3, COL5A2, COL11A1, FN1, MMP2, CTGF, TGFB2, and/or TGFB3. In some embodiments, administration of a miR-29 mimic down-regulates inflammatory responses associated with fibrosis. In some embodiments, administration of a miR-29 mimic reduces the levels of pro-inflammatory cytokines such as IL-12, IL-4, GCSF, and TNF-α in fibrosis patients. In some embodiments, administration of a miR-29 mimic reduces infiltration of immune effector cells such as neutrophils, lymphocytes, monocytes, and macrophages in fibrotic tissues or organs. In some embodiments, administration of a miR-29 mimic reduces or inhibits epithelial-to-mesenchymal transition. In some embodiment, administration of miR-29 mimic reduces or inhibits myofibroblast differentiation.

In certain embodiments, the present disclosure provides methods of regulating an extracellular matrix gene in an ocular cell comprising contacting the cell with a miR-29 mimic of the present disclosure. In some embodiments, the disclosure provides methods of regulating a collagen synthesis gene in an ocular cell comprising contacting the ocular cell with a miR-29 mimic of the present disclosure. Upon treatment or contact, the miR-29 mimic reduces the expression or activity of the extracellular matrix gene or the collagen synthesis gene.

As used herein, the term “subject” or “patient” refers to any vertebrate including, without limitation, humans and other primates (e.g., chimpanzees and other apes and monkey species), farm animals (e.g., cattle, sheep, pigs, goats and horses), domestic mammals (e.g., dogs and cats), laboratory animals (e.g., rabbits, rodents such as mice, rats, and guinea pigs), and birds (e.g., domestic, wild and game birds such as chickens, turkeys and other gallinaceous birds, ducks, geese, and the like). In some embodiments, the subject is a mammal. In other embodiments, the subject is a human.

In some embodiments, the subject suffers from diabetes. In some embodiments, the subject suffers from diabetic retinopathy. In some embodiments, the subject suffers from age-related macular degeneration (AMD). In some embodiments, the subject suffers from wet age-related macular degeneration (wet AMD). In some embodiments, the subject is being treated for the diabetic retinopathy with an anti-VEGF therapy that gives rise to retinal fibrosis. In some embodiments, the disclosure provides a method of treating diabetic retinopathy and/or the retinal fibrosis associated with the treatments used for the diabetic retinopathy in a subject in need thereof comprising administering a miR-29 mimic of the disclosure to the subject. In some embodiments, the disclosure provides a method of treating diabetic retinopathy in a subject in need thereof comprising administering a miR-29 mimic of the disclosure to the subject while also administering an anti-VEGF therapy to the subject simultaneously, sequentially, or intermittently. In some embodiments, a miR-29 mimic and an anti-VEGF therapy are co-administered by intravitreal injection. Treatment of diabetic retinopathy with an anti-VEGF treatment may require repeated injection of the anti-VEGF treatment. Co-administration of a miR-29 mimic of the invention with the anti-VEGF therapy may inhibit, lessen, or prevent damage to the eye (including but not limited to ocular fibrosis) due to single or repeated injection of the anti-VEGF treatment. In some embodiments, the miR-29 mimic of the disclosure is co-administered with other ocular therapeutics. Co-administration of a miR-29 mimic of the invention with the ocular therapeutic may inhibit, lessen, or prevent damage to the eye (including but not limited to ocular fibrosis) due to single or repeated injection of the ocular therapeutic. Exemplary anti-VEGF treatments of the disclosure include, with limitation, anti-VEGF antisera, soluble VEGF receptor, anti-VEGF aptamers, VEGFR1-neutralizing antisera, anti-VEGF polyclonal antibodies, anti-VEGF monoclonal antibodies, and anti-VEGF bispecific or multispecific antibodies. Further exemplary anti-VEGF treatments include, with limitation, bevacizumab (Avastin), which may be combined with fluorouracil and leucovorin; ranibizumab (Lucentis), aflibercept, ziv-aflibercept, or brolucizumab.

In some embodiments, the disclosure provides methods of treating, preventing, decreasing, or diminishing ocular fibrosis secondary to treatment of the eye with an antibody, a small-molecule drug, a biologic drug, an aptamer, or a virus (e.g. a viral vector). In some embodiments, the disclosure provides a method of performing eye surgery comprising administering a miR-29 mimic before, during, or after the eye surgery. In some embodiments, the eye surgery comprises laser eye surgery (e.g. LASIK). In some embodiments, the eye surgery comprises refractive surgery for the correction of myopia, hyperopia, and astigmatism. In some embodiments, the eye surgery comprises photorefractive keratectomy (PRK). In some embodiments, the eye surgery comprises retinal detachment repair. Administration of a miR-29 mimic of the disclosure reduces side-effects of eye surgery, including without limitation higher-order aberrations, dry eyes, halos, diffuse lamellar keratitis, corneal scarring, epithelial in-growth, choroidal neovascularization, uveitis, and eye floaters. In some embodiments, the disclosure provides a method of treating eye injury comprising administering a miR-29 mimic with 1, 2, 5, 10, or 24 hours of the eye injury. In some embodiments, the eye injury is a traumatic or accidental eye injury.

The disclosure also provides methods for assessing the efficacy of an ocular fibrosis treatment with a miR-29 agonist (e.g., drug or miR-29 mimic). For instance, in some embodiments, the method for assessing the treatment efficacy comprises determining a level of expression of one or more genes in ocular cells of a subject prior to the treatment with a miR-29 mimic, wherein the one or more genes are selected from a set of genes modulated by miR-29, determining the level of expression of the same one or more genes in cells/fibrotic tissue of the subject after treatment with a miR-29 mimic; and determining the treatment to be effective, less effective, or not effective based on the expression levels prior to and after the treatment. In another embodiment, at least 1-fold, 1.5-fold, 2-fold, 2.5-fold, 3-fold, 3.5-fold, or 4-fold difference in the expression of the genes prior to and after treatment indicates the treatment to be effective.

Pharmaceutical Compositions

The present disclosure also provides pharmaceutical compositions comprising a therapeutically effective amount of one or more microRNA mimetic compounds of miR-29a, miR-29b, and/or miR-29c according to the disclosure or a pharmaceutically acceptable salt thereof, and a pharmaceutically acceptable carrier or excipient.

In some embodiments, the pharmaceutical composition comprises a therapeutically effective amount of a miR-29a mimetic compound and a pharmaceutically acceptable carrier or excipient, wherein the first strand of the mimetic compound comprises a mature miR-29a sequence and the second strand is substantially complementary to the first strand. In another embodiment, the pharmaceutical composition comprises a therapeutically effective amount of a miR-29b mimetic compound and a pharmaceutically acceptable carrier or excipient, wherein the first strand of the mimetic compound comprises a mature miR-29b sequence and the second strand is substantially complementary to the first strand. In yet another embodiment, the pharmaceutical composition comprises a therapeutically effective amount of a miR-29c mimetic compound and a pharmaceutically acceptable carrier or excipient, wherein the first strand of the mimetic compound comprises a mature miR-29c sequence and the second strand is substantially complementary to the first strand.

In some embodiments, the pharmaceutical composition comprises a therapeutically effective amount of at least two microRNA mimetic compounds of the disclosure and a pharmaceutically acceptable carrier or excipient. For instance, a pharmaceutical composition may comprise a combination of a miR-29a and a miR-29b mimics; a miR-29a and a miR-29c mimics; or a miR-29b and a miR-29c mimics. Alternatively, the composition may comprise two mimics of the same microRNA. In yet some other embodiments, the disclosure provides pharmaceutical compositions comprising a therapeutically effective amount of three microRNA mimetic compounds of the disclosure and a pharmaceutically acceptable carrier or excipient. For instance, a pharmaceutical composition may comprise a combination of a miR-29a, a miR-29b and a miR-29c mimics.

In some embodiments where the pharmaceutical compositions comprise at least two microRNA mimetic compounds according to the disclosure, the first and the second mimetic compounds or the first, second and the third mimetic compounds may be present in equimolar concentrations. Other mixing ratios such as about 1:2, 1:3, 1:4, 1:5, 1:2:1, 1:3:1, 1:4:1, 1:2:3, 1:2:4 are also envisioned for preparing pharmaceutical compositions comprising at least two of the miR-29a, miR-29b, and miR-29c mimetic compounds.

The active compounds and compositions of the present disclosure may include classic pharmaceutical preparations. Administration of these compositions according to the present disclosure may be via any common route so long as the target tissue is available via that route.

In another embodiment of the disclosure, compositions comprising a miR-29 mimic as described herein may be formulated as a coating for a medical device, such as a contact lens, sponge, or other material.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above.

The compositions of the present disclosure generally may be formulated in a neutral or salt form. Pharmaceutically-acceptable salts include, for example, acid addition salts (formed with the free amino groups of the protein) derived from inorganic acids (e.g., hydrochloric or phosphoric acids), or from organic acids (e.g., acetic, oxalic, tartaric, mandelic, and the like). Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like).

Upon formulation, solutions are preferably administered in a manner compatible with the dosage formulation and in such amount as is therapeutically effective. The formulations may easily be administered in a variety of dosage forms such as injectable solutions, topical solutions, drug-eluting devices or other coated vascular devices, and the like. In specific embodiments, the miR-29 mimetic compound is formulated for administration via iontophoresis, subconjunctival injection, injection into the pterygium, injection at site of a trabecular bleb, sub-tendon injection, an intravitreal injection, intracamerally, or topically.

Colloidal dispersion systems, such as macromolecule complexes, nanocapsules, microspheres, beads, and lipid-based systems including oil-in-water emulsions, micelles, mixed micelles, liposomes and exosomes, may be used as delivery vehicles for miR-29a, miR-29b, and/or miR-29c mimetic compounds. In some embodiments, the miR-29 mimic of the present disclosure may be formulated into liposome particles.

Commercially available fat emulsions that are suitable for delivering the nucleic acids of the disclosure to target tissues include Intralipid®, Liposyn®, Liposyn® II, Liposyn® III, Nutrilipid, and other similar lipid emulsions. A preferred colloidal system for use as a delivery vehicle in vivo is a liposome (i.e., an artificial membrane vesicle). The preparation and use of such systems is well known in the art. Exemplary formulations are also disclosed in U.S. Pat. No. 5,981,505; U.S. Pat. No. 6,217,900; U.S. Pat. No. 6,383,512; U.S. Pat. No. 5,783,565; U.S. Pat. No. 7,202,227; U.S. Pat. No. 6,379,965; U.S. Pat. No. 6,127,170; U.S. Pat. No. 5,837,533; U.S. Pat. No. 6,747,014; and WO03/093449, which are herein incorporated by reference in their entireties.

In certain embodiments, liposomes used for delivery are amphoteric liposomes such SMARTICLES® (Marina Biotech, Inc.) which are described in detail in U.S. Pre-grant Publication No. 20110076322. The surface charge on the SMARTICLES® is fully reversible which make them particularly suitable for the delivery of nucleic acids. SMARTICLES® can be delivered via injection, remain stable, and aggregate free and cross cell membranes to deliver the nucleic acids.

In some embodiments, microRNA mimetic compounds or pharmaceutical compositions of the disclosure are formulated for ocular delivery and can be in the form of powders, aqueous solutions, or ocular drops. Solid formulations may contain excipients such as lactose or dextran. Liquid formulations may be aqueous or oily solutions for use in the form of aerosols, drops or metered spray.

In some embodiments, formulations are a liposomal formulation, a nano-suspension formulation, or a microsuspension formulation.

In some embodiments, the pharmaceutical composition comprises a therapeutically effective amount of a miR-29a mimetic compound and a pharmaceutically acceptable carrier, wherein the pharmaceutically acceptable carrier is selected from water for injection (WFI), 0.9% (w/v) sodium chloride, 5 mM phosphate buffer, 10 mM phosphate buffer, 25 mM phosphate buffer, 50 mM phosphate buffer, 85 mM phosphate buffer, and 100 mM phosphate buffer. In some embodiments, the pharmaceutical composition comprises a therapeutically effective amount of a miR-29a mimetic compound and a pharmaceutically acceptable carrier, wherein the pharmaceutically acceptable carrier is selected from water for injection (WFI), 10 mM phosphate buffer, and 85 mM phosphate buffer.

In some embodiments, the pharmaceutical composition comprises a therapeutically effective amount of a miR-29b mimetic compound and a pharmaceutically acceptable carrier, wherein the pharmaceutically acceptable carrier is selected from water for injection (WFI), 0.9% (w/v) sodium chloride, 5 mM phosphate buffer, 10 mM phosphate buffer, 25 mM phosphate buffer, 50 mM phosphate buffer, 85 mM phosphate buffer, and 100 mM phosphate buffer. In some embodiments, the pharmaceutical composition comprises a therapeutically effective amount of a miR-29b mimetic compound and a pharmaceutically acceptable carrier, wherein the pharmaceutically acceptable carrier is selected from water for injection (WFI), 10 mM phosphate buffer, and 85 mM phosphate buffer.

In some embodiments, the pharmaceutical composition comprises a therapeutically effective amount of a miR-29c mimetic compound and a pharmaceutically acceptable carrier, wherein the pharmaceutically acceptable carrier is selected from water for injection (WFI), 0.9% (w/v) sodium chloride, 5 mM phosphate buffer, 10 mM phosphate buffer, 25 mM phosphate buffer, 50 mM phosphate buffer, 85 mM phosphate buffer, and 100 mM phosphate buffer. In some embodiments, the pharmaceutical composition comprises a therapeutically effective amount of a miR-29c mimetic compound and a pharmaceutically acceptable carrier, wherein the pharmaceutically acceptable carrier is selected from water for injection (WFI), 10 mM phosphate buffer, and 85 mM phosphate buffer.

In some embodiments, the pharmaceutical composition comprises 0.2 mg/mL to 200 mg/mL of the miR-29 mimic. In some embodiments, the pharmaceutical composition comprises 0.2 mg/mL to 10 mg/mL, 10 mg/mL to 50 mg/mL, 50 mg/mL to 90 mg/mL, or 90 mg/mL to 120 mg/mL of the miR-29 mimic. In some embodiments, the pharmaceutical composition comprises 0.2 mg/mL to 1 mg/mL, 1 mg/mL to 5 mg/mL, 5 mg/mL to 10 mg/mL, 10 mg/mL to 20 mg/mL, 20 mg/mL to 30 mg/mL, 30 mg/mL to 40 mg/mL, 40 mg/mL to 50 mg/mL, 50 mg/mL to 60 mg/mL, 60 mg/mL to 70 mg/mL, 70 mg/mL to 90 mg/mL, or 90 mg/mL to 120 mg/mL of the miR-29 mimic. In some embodiments, the pharmaceutical composition comprises at least about 0.3 mg/mL, at least about 0.5 mg/mL, at least about 1 mg/mL, at least about 2 mg/mL, at least about 3 mg/mL, at least about 4 mg/mL, at least about 5 mg/mL, at least about 7 mg/mL, at least about 10 mg/mL, at least about 20 mg/mL, at least about 30 mg/mL, at least about 40 mg/mL, or at least about 50 mg/mL of the miR-29 mimic.

In some embodiments, the pharmaceutical composition comprises about 100 mg/mL, about 70 mg/mL, about 35 mg/mL, about 7 mg/mL, about 3.5 mg/mL, about 0.7 mg/mL, or 0.35 mg/mL of the miR-29 mimic. In some embodiments, the pharmaceutical composition comprises about 70 mg/mL of the miR-29 mimic. In some embodiments, the pharmaceutical composition comprises about 35 mg/mL of the miR-29 mimic.

In some embodiments, the pharmaceutical composition has a pH of 4 to 9. In some embodiments, the pharmaceutical composition has a pH of about 4, about 4.5, about 5, about 5.5, about 6, about 6.5, about 7, about 7.5, about 8, about 8.5, or about 9.

In some embodiments, the pharmaceutical composition is formulated as a combination therapy. In some embodiments, the pharmaceutical composition comprises a miR-29 mimic of the disclosure and one or more anti-VEGF treatments, including without limitation one or more of bevacizumab (Avastin), ranibizumab (Lucentis), aflibercept, ziv-aflibercept, or brolucizumab. In some embodiments, the combination therapy is an ophthalmic formulation. In some embodiments, the combination therapy is formulated for intravitreal injection. In some embodiments, the combination therapy further comprises one or both of fluorouracil and leucovorin. In some embodiments, the pharmaceutical compositions comprises a miR-29 mimic of the disclosure and one or more anti-VEGF small-molecule drugs, e.g. PF-00337210.

In some embodiments, the pharmaceutical composition comprises a miR-29 mimic of the disclosure and one or more cancer therapeutics. For example, the disclosure provides a pharmaceutical composition comprising a miR-29 mimic of the disclosure and rituximab formulated. In some embodiments, the disclosure provides a pharmaceutical composition for use in treating or preventing intraocular lymphoma.

In some embodiments, the pharmaceutical composition comprises an antibiotic. An antibiotic may be provided as a three-drug combination with an anti-VEGF treatment and a miR-29 mimic of the disclosure for prophylaxis of endophthalmitis or other infectious diseases of the eye. In some embodiments, the disclosure provides a pharmaceutical composition comprising a miR-29 mimic of the disclosure and one or more of antibiotic, antifungal, antimicrobial, or an antiviral.

In some embodiments, the pharmaceutical compositions of the disclosure comprise 2, 3, 4, 5, 6, 7, 8, 9, 10 or more miR-29 mimics of the disclosure. In some embodiments, the pharmaceutical composition comprises one or more miR-29 mimics of the disclosure and one or more other microRNAs or microRNA mimics, including without limitation miR-29 mimics other than those disclosed herein.

In some embodiments, the disclosure provides a pharmaceutical composition for use in subjects undergoing treatment for diabetic retinopathy, AMD, or wet AMD to be administered regularly before, during, or after injection of a therapeutic agent into the eye. A miR-29 mimic of the disclosure or a pharmaceutical composition comprising a miR-29 mimic of the disclosure may be supplied in a kit for medical-office or home use, optionally as an eye drop, optionally accompanied by instructions for use.

A skilled artisan may desire to employ appropriate salts and buffers to render delivery vehicles stable and allow for uptake by target ocular cells. Aqueous compositions of the present disclosure comprise an effective amount of the delivery vehicle comprising the miR-29 mimic (e.g. liposomes or other complexes) dissolved or dispersed in a pharmaceutically acceptable carrier or aqueous medium. The phrases “pharmaceutically acceptable” or “pharmacologically acceptable” refers to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present disclosure, its use in therapeutic compositions is contemplated. Supplementary active ingredients also can be incorporated into the compositions, provided they do not inactivate the polynucleotides of the compositions.

Administration and Dosing

As provided herein, one or more miR-29 mimetic compounds are administered for the treatment of ocular fibrosis. In some embodiments, the miR-29 mimetic compound is directly administered in to the eye/ocular cells of the subject. In some embodiments, the miR-29 mimetic compound is administered peripherally. In some embodiments, the miR-29 mimetic compound is administered via iontophoresis, subconjunctival injection, injection into the pterygium, injection at site of a trabecular bleb, sub-tendon injection, an intravitreal injection, intracamerally, or topically.

In some embodiments, the pharmaceutical composition comprises a drop, ointment, or suspension. In some embodiments, the disclosure provides a method of administering a miR-29 mimic or pharmaceutical composition comprising a miR-29 mimic, comprising a route of administration selected from: topical administration, contacting the eye with a drug-eluting contact lens, sub-conjunctival injection, intra-bleb injection, intracameral injection, intracameral depot (e.g., drug-embedded “seed” of silicon or other material), intravitreal injection, intravitreal depot (e.g., drug-embedded “seed” of silicon or other material), and iontophoresis.

In some embodiments, the disclosure provides a method of treating corneal fibrosis, comprising administering a miR-29 mimic or pharmaceutical composition comprising a miR-29 mimic, comprising topical administration or contacting the eye with a drug-eluting contact lens. In some embodiments, the pharmaceutical composition is a drop.

In some embodiments, the disclosure provides a method of treating pterygium, comprising administering a miR-29 mimic or pharmaceutical composition comprising a miR-29 mimic, comprising topical administration or sub-conjunctival injection. In some embodiments, the pharmaceutical composition is a drop.

In some embodiments, the disclosure provides a method of treating trabeculectomy bleb, comprising administering a miR-29 mimic or pharmaceutical composition comprising a miR-29 mimic, comprising intra-bleb injection, sub-conjunctival injection, or topical administration. In some embodiments, the pharmaceutical composition is a drop.

In some embodiments, the disclosure provides a method of treating glaucoma, comprising administering a miR-29 mimic or pharmaceutical composition comprising a miR-29 mimic, comprising sub-conjunctival injection, intracameral injection, or intracameral depot. In some embodiments, the intracameral depot comprises a drug-embedded “seed” of silicon or other pharmaceutically acceptable material.

In some embodiments, the disclosure provides a method of treating retinal fibrosis, comprising administering a miR-29 mimic or pharmaceutical composition comprising a miR-29 mimic, comprising intravitreal injection, intravitreal depot, or iontophoresis. In some embodiments, the intravitreal depot comprises a drug-embedded seed of silicon or other pharmaceutically acceptable material.

In some embodiments, one or more microRNA mimetic compounds of the disclosure may be administered concurrently but in separate compositions, with concurrently referring to mimetic compounds given within a short period, for instance, within about 5, 10, 20, or 30 minutes of each other. In some other embodiments, miR-29a, miR-29b, and/or miR-29c mimetic compounds may be administered in separate compositions at different times.

The disclosure also encompasses embodiments where additional therapeutic agents may be administered along with miR-29a, miR-29b, and/or miR-29c mimetic compounds. In some embodiments, the additional therapeutic agent is a second anti-fibrotic agent. The additional therapeutic agents may be administered concurrently but in separate formulations or sequentially. In other embodiments, additional therapeutic agents may be administered at different times prior to after administration of miR-29a, miR-29b, and/or miR-29c mimetic compounds. Where clinical applications are contemplated, pharmaceutical compositions will be prepared in a form appropriate for the intended application. Generally, this can entail preparing compositions that are essentially free of pyrogens, as well as other impurities that could be harmful to humans or animals.

In some exemplary embodiments, the miR-29 mimetic compound is administered topically into the cornea. In some exemplary embodiments, the miR-29 mimetic compound is administered topically into the cornea, for example in a drop formulation. In some embodiments, the miR-29 mimetic compound is administered topically into the cornea, for example twice daily, or for example four times daily. In some embodiments, the miR-29 mimetic compound is administered topically into the cornea, for example four times daily.

In some exemplary embodiments, the miR-29 mimetic compound is administered topically periodically for up to one week. In some exemplary embodiments, the miR-29 mimetic compound is administered topically periodically for up to four weeks. In some exemplary embodiments, the miR-29 mimetic compound is administered topically twice daily. In some embodiments, the miR-29 mimetic compound is administered topically twice daily for up to four weeks. In some exemplary embodiments, the miR-29 mimetic compound is administered topically periodically for up to 28 days. In some embodiments, the miR-29 mimetic compound is administered topically twice daily for up to 28 days. It will be understood that throughout the disclosure the terms once daily, twice daily, etc. refer to the frequency of unilateral or bilateral administration, i.e. “twice daily” refers to either in one eye two times in a day or in two eyes two times in a day, and not to administration in both eyes one time per day. Generally the composition of the disclosure are administered bilaterally when treatment of both eyes is desired and unilaterally when treatment of only one eye is desired—e.g., for treatment of eye injury in only the left eye, the compositions of the disclosure are administered only to the left right.

In some exemplary embodiments, the miR-29 mimetic compound is administered in a drop formulation.

In some exemplary embodiments, the miR-29 mimetic compound is administered via intravitreal injection once weekly.

In some exemplary embodiments, the miR-29 mimetic compound is administered via intravitreal injection periodically for one month.

The disclosure further provides vectors, pharmaceutical compositions, and methods for administering a miR-29 mimic by viral gene delivery. In some embodiments, the viral vector comprises a lentiviral vector, an adenoviral vector, or an adeno-associated virus (AAV) vector. Delivery of a miR-29 mimic to the human body via an AAV vector is described, for example, in Heller et al. JCI Insight. 2017 May 4; 2(9): e93309, and International Patent Publication No. WO2017181011A1, each of which is incorporated herein in its entirety. AAV vectors can be adapted for use in the eye by, e.g., use of serotypes for improved ocular gene transfer as described in Lebherz et al. J Gene Med. 2008 Apr; 10(4): 375-382.

The disclosure further provides kits comprising therapeutic mir-29 mimics and compositions described herein. The present disclosure also provides articles of manufacture comprising any one of the therapeutic compositions or kits described herein. Examples of an article of manufacture include vials (e.g. sealed vials).

### ENUMERATED EMBODIMENTS

### Embodiment 1

A method of treating ocular fibrosis in a subject comprising administering to the subject a therapeutically effective amount of a miR-29 mimetic compound, wherein the miR-29 mimetic compound comprises:

(a) a first strand of 23 to 26 ribonucleotides comprising a mature miR-29a, miR-29b, or miR-29c sequence; and

(b) a second strand of 22 to 23 ribonucleotides comprising a sequence that is substantially complementary to the first strand and having at least one modified nucleotide, wherein the first strand has a 3′ nucleotide overhang relative to the second strand,

wherein the ocular fibrosis comprises retinal fibrosis, corneal fibrosis, conjunctival fibrosis, or fibrosis of the trabecular meshwork.

### Embodiment 2

The method of embodiment 1, wherein the ocular fibrosis results from an ocular disease or disorder selected from the group consisting of diabetic retinopathy, bacterial infection, viral infection, fungal infection, amoeba infection, ocular trauma, chemical corneal burn, thermal corneal burn, pterygium, glaucoma, surgical trauma, Fuch's Endothelial Corneal Dystrophy (FECD), and anterior proliferative vitreoretinopathy (anterior PVR).

### Embodiment 3

A method of treating an ocular disease or disorder in a subject comprising administering to the subject a therapeutically effective amount of a miR-29 mimetic compound, wherein the miR-29 mimetic compound comprises:

(a) a first strand of 23 to 26 ribonucleotides comprising a mature miR-29a, miR-29b, or miR-29c sequence; and

(b) a second strand of 22 to 23 ribonucleotides comprising a sequence that is substantially complementary to the first strand and having at least one modified nucleotide, wherein the first strand has a 3′ nucleotide overhang relative to the second strand,

wherein the ocular disease or disorder is selected from the group consisting of diabetic retinopathy, bacterial infection, viral infection, fungal infection, amoeba infection, ocular trauma, chemical corneal burn, thermal corneal burn, pterygium, glaucoma, surgical trauma, Fuch's Endothelial Corneal Dystrophy (FECD), and anterior proliferative vitreoretinopathy (anterior PVR).

### Embodiment 4

The method of embodiment 3, wherein the ocular disease or disorder comprises an ocular fibrosis selected from the group consisting of comprises retinal fibrosis, corneal fibrosis, conjunctival fibrosis, or fibrosis of the trabecular meshwork

### Embodiment 5

The method of any one of embodiments 1 to 4, wherein the miR-29 mimetic compound is administered via iontophoresis, subconjunctival injection, injection into the pterygium, injection at site of a trabecular bleb, sub-tendon injection, an intravitreal injection, intracamerally, or topically.

### Embodiment 6

The method of any one of embodiments 1, 2 or 4, wherein the ocular fibrosis comprises proliferative vitreoretinopathy.

### Embodiment 7

The method of any one of embodiments 1, 2 or 4, wherein the ocular fibrosis comprises retinal fibrosis associated with diabetic retinopathy.

### Embodiment 8

The method of embodiment 7, wherein the ocular fibrosis comprises retinal fibrosis associated with anti-VEGF treatment.

### Embodiment 9

The method of any one of embodiments 1, 2 or 4, wherein the ocular fibrosis comprises corneal hazing or corneal scarring.

### Embodiment 10

The method of any one of embodiments 1 to 4, wherein the ocular fibrosis results from a corneal burn.

### Embodiment 11

The method of any one of embodiments 1 to 4, wherein the ocular fibrosis results from pterygium.

### Embodiment 12

The method of any one of embodiments 1 to 4, wherein the ocular fibrosis results from Fuch's Endothelial Corneal Dystrophy (FECD).

### Embodiment 13

The method of any one of embodiments 1 to 4, wherein the ocular fibrosis results from glaucoma.

### Embodiment 14

The method of any one of embodiments 1 to13, wherein the ocular fibrosis comprises a trabecular bleb.

### Embodiment 15

The method of any one of embodiments 1 to 14, wherein the miR-29 mimetic compound is administered topically into the cornea.

### Embodiment 16

The method of embodiment 15, wherein the miR-29 mimetic compound is administered topically into the cornea in a drop formulation.

### Embodiment 17

The method of any one of embodiments 15 to 16, wherein the miR-29 mimetic compound is administered topically into the cornea once daily, twice daily, three times daily, or four times daily.

### Embodiment 18

The method of any one of embodiments 15 to 16, wherein the miR-29 mimetic compound is administered topically periodically for up to one week, two weeks, three weeks, four weeks, or 28 days.

### Embodiment 19

The method of any one of embodiments 1 to 14, wherein the miR-29 mimetic compound is administered in a drop formulation.

### Embodiment 20

The method of any one of embodiments 1 to 14, wherein the miR-29 mimetic compound is administered via intravitreal injection once weekly.

### Embodiment 21

The method of any one of embodiments 1 to 14, wherein the miR-29 mimetic compound is administered via intravitreal injection periodically for one month.

### Embodiment 23

The method of any one of embodiments 1 to 22, wherein the miR-29 mimetic compound is presented in Table 2.

### Embodiment 22

The method of any one of embodiments 1 to 21, wherein the expression of COL1A1, COL1A2, COL3A1, COL4A3, COL5A2, COL11A1, FN1, MMP2, CTGF, TGFB2, and/or TGFB3 is reduced.

### Embodiment 24

The method of any one of embodiments 1 to 22, wherein the first strand comprises a sequence selected from SEQ ID NO: 2, 18-21, or 31-34 and the second strand comprises a sequence selected from 1, 8-10, 13-17, or 28-30.

### Embodiment 25

The method of any one of embodiments 1 to 22, wherein the miR-29 mimetic compound comprises:

(a) a first strand of about 23 to about 26 ribonucleotides comprising a mature miR-29b sequence, wherein the first strand comprises a sequence selected from SEQ ID NO: 2, 18-21, and 31-34; and

(b) a second strand of about 22 to about 25 ribonucleotides comprising a sequence that is substantially complementary to the first strand and having at least one modified nucleotide, wherein the first strand has a 3′ nucleotide overhang relative to the second strand.

### Embodiment 26

The method of any one of embodiments 1 to 22, wherein the miR-29 mimetic compound comprises:

(a) a first strand of about 23 to about 26 ribonucleotides comprising a mature miR-29b sequence; and

(b) a second strand of about 22 to about 25 ribonucleotides comprising a sequence that is substantially complementary to the first strand and having at least one modified nucleotide, wherein the first strand has a 3′ nucleotide overhang relative to the second strand, and wherein the second strand comprises a sequence selected from SEQ ID NO: 1, 8-10, 13-17, and 28-30.

### Embodiment 27

The method of any one of embodiments 1 to 22, wherein the first strand comprises the sequence of SEQ ID NO: 19 and the second strand comprises the sequence of SEQ ID NO: 1.

### Embodiment 28

The method of any one of embodiments 1 to 22, wherein the first strand comprises the sequence of SEQ ID NO: 19 and the second strand comprises the sequence of SEQ ID NO: 15.

### Embodiment 29

The method of any one of embodiments 1 to 22, wherein the first strand comprises a mature miR-29a sequence.

### Embodiment 30

The method of any one of embodiments 1 to 22, wherein the miR-29 mimetic compound is presented in Table 1.

### Embodiment 31

The method of any one of embodiments 1 to 22, wherein the first strand comprises a sequence of SEQ ID NO: 6, 7 or 27 and the second strand comprises a sequence selected from SEQ ID NO: 3-5 or 11.

### Embodiment 32

The method of any one of embodiments 1 to 22, wherein the first strand comprises a mature miR-29c sequence.

### Embodiment 33

The method of any one of embodiments 1 to 22, wherein the miR-29 mimetic compound is presented in Table 3.

### Embodiment 34

The method of any one of embodiments 1 to 22, wherein the first strand comprises a sequence of SEQ ID NO: 25, 26, or 35 and the second strand comprises a sequence selected from SEQ ID NO: 22-24 or 12.

### Embodiment 35

The method of any one of embodiments 1 to 34, wherein a therapeutically effective amount of the miR-29 mimetic compound is comprised in a pharmaceutical composition.

### Embodiment 36

The method of any one of embodiments 1 to 35, wherein the subject is a human.

### Embodiment 37

The method of embodiment 36, wherein the subject suffers from diabetes.

### Embodiment 38

The method of embodiment 36 or 37, wherein the subject suffers from diabetic retinopathy.

### Embodiment 39

The method of embodiment 36 or 37, wherein the subject suffers from age-related macular degeneration (AMD).

### Embodiment 40

The method of embodiment 39, wherein the subject suffers from wet age-related macular degeneration (wet AMD).

### Embodiment 41

The method of any one of embodiments 1 to 40, wherein the subject is receiving an anti-VEGF therapy.

This disclosure is further illustrated by the following additional examples that should not be construed as limiting. Those of skill in the art should, in light of the present disclosure, appreciate that many changes can be made to the specific embodiments which are disclosed and still obtain a like or similar result without departing from the spirit and scope of the disclosure.

All patent and non-patent documents referenced throughout this disclosure are incorporated by reference herein in their entirety for all purposes.

### EXAMPLES

### Example 1: Introduction of a miR-29b mimic Following Corneal Alkali Burn

Alkali burn representative of damage to the cornea. It is representative, inter alia, of the epithelium breakdown induced by viral infection, bacterial infection, fungal infection, neurotrophic keratitis, sterile keratitis, corneal trauma, and surgery. It is known that alkali burn has a reliable time course with upregulation of extracellular matrix molecule expression at 7-14 days post-burn and corneal hazing and scarring apparent both grossly and histologically at 10-14 days post-burn.

Methods

Alkali Burn: 8-10 week old male Sprague Dawley rats were anesthetized with isoflurane and 0.5% Proparacaine Hydrochloride Ophthalmic Solution was applied for local anesthesia. One cornea per animal was subjected to an alkali burn by application of 0.5 or 1N NaOH for 10 seconds, followed by saline rinse. Antibiotic ointment was applied twice daily thereafter to prevent infection. Alkali burn was confirmed in a subset of animals by fluorescein staining at the time of burn and/or by histology (hematoxylin and eosin staining) 6 hours after burn creation.

miR-29b mimic Topical Administration: Saline or double-stranded miR-29b mimic (SEQ ID NO: 1/SEQ ID NO: 19) was administered topically to the cornea of rats in drop formulation (10 uL per drop, 1 or 70 mg/mL miR-29b mimic in sterile saline). Drops were applied four times daily for 1 day or twice daily for one to 28 days. Uptake/distribution of a fluorescein isothiocyanate (FITC)-labeled oligo was assessed via confocal microscopy.

miR-29 Target Expression: Total RNA was extracted from alkali burned rat corneas treated with saline or miR-29b mimic treatment for 7, 10 or 14 days using Trizol (Life Technologies). Expression of miR-29 targets and miR-29b mimic pharmacodynamic biomarkers (COL1A1, COL1A2, COL5A2, COL11A1, MMP2, TGFB2, and/or FN1) was assessed via qRT-PCR at Days 7, 10, and 14 (reagents from Life Technologies). Pharmacokinetics was assessed by quantitative RT-PCR from whole tissue homogenates over time.

Histopathology: Alkali burned corneas treated with saline or miR-29b mimic for 4, 7, 10 or 14 days were formalin fixed and paraffin embedded. Thickness of the corneal epithelium and stroma were assessed using 20× images captured from hematoxylin and eosin stained sections (using Aperio ImageScope software), and alpha-smooth muscle actin (α-SMA) positive myofibroblasts were quantified from slides stained with an anti-α-SMA antibody and colorimetric substrate with hematoxylin counterstain (analysis performed in ImageJ software).

Clinical assessment: Alkali burned corneas treated with saline or miR-29b mimic for 10, 14, 21 or 28 days were assessed clinically for corneal haze or corneal scarring using a 1-5 scale whereby 1 represented a normal, clear cornea and 5 represented a cornea with severe scarring and/or hazing (1=absent, 2=minimal, 3=mild, 4=moderate, 5=severe).

These methods were repeated across multiple studies.

Results

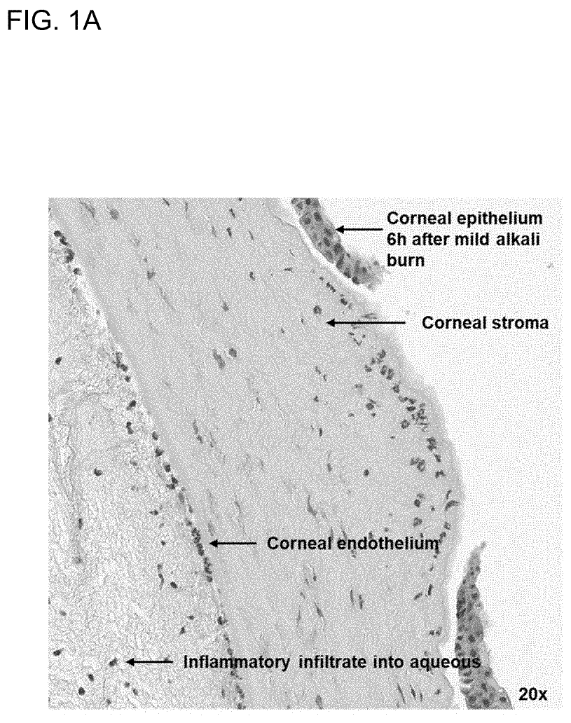

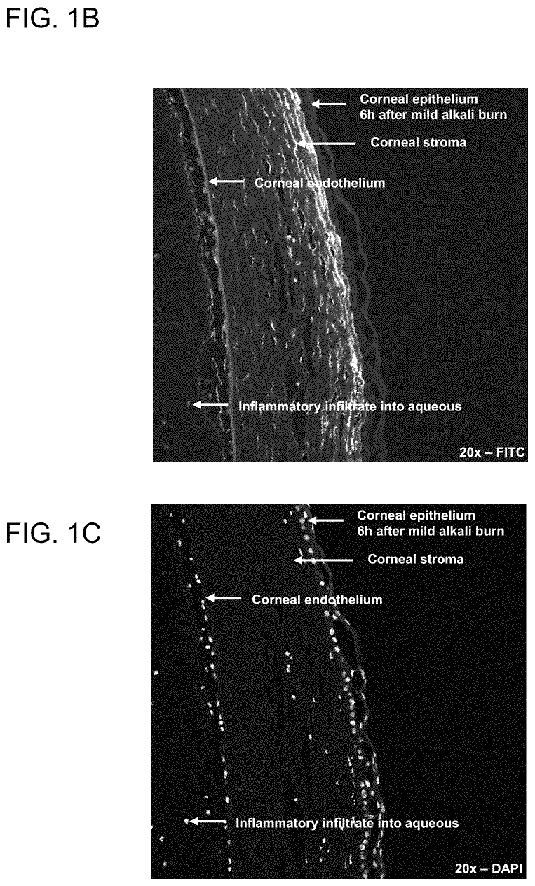

An alkali burn was created in one eye per rat by administration of 1N NaOH (FIG. 1A). Topically applied FITC-miR-29b mimic (1 mg/mL) was taken up into all layers of the alkali burned rat cornea and stains the corneal stroma and corneal endothelium most strongly (FIG. 1B). DAPI nuclear stain of the same view is also shown (FIG. 1C).

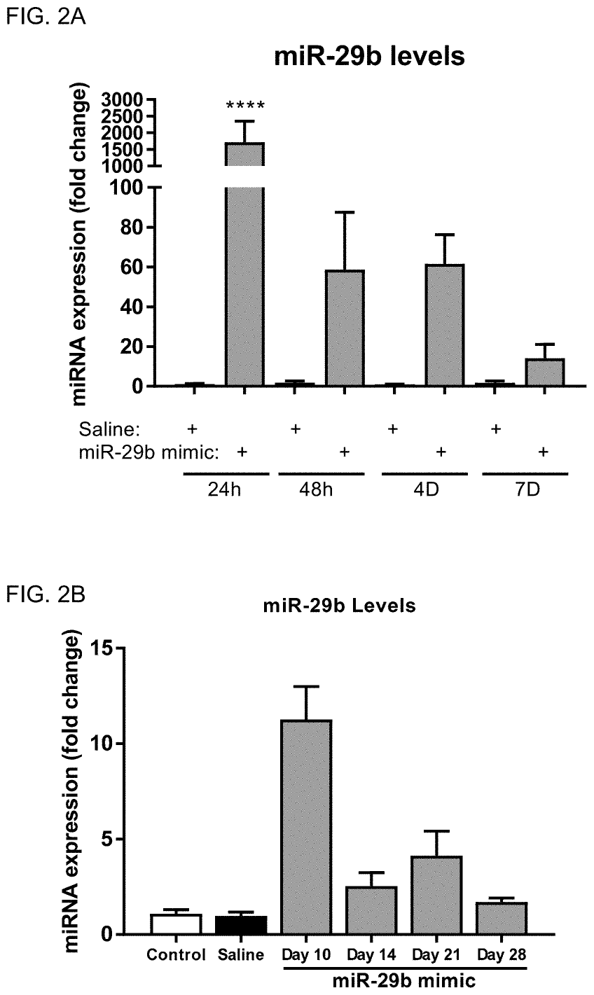

Saline or miR-29b mimic (70 mg/mL) was administered twice daily to rats via topical (drop) application after creation of the corneal alkali burn, for 1 to 28 days. Eyes were harvested 6 hours after the last drop application and miR-29b was quantified by qRT-PCR. Endogenous miR-29b levels were largely unchanged after burn. Conversely, miR-29b mimic treated eyes show substantial increases in miR-29b levels following miR-29b mimic treatment, with the greatest uptake being seen immediately after burn creation (1717-fold above endogenous) and less uptake being seen after re-establishment of the corneal epithelium (14-fold above endogenous at day 7) (FIG. 2A). In a separate study, additional time points were assessed (FIG. 2B), with similar results (miR-29b levels detected 11-fold above endogenous at day 10 and 1.7-fold above endogenous at day 28).

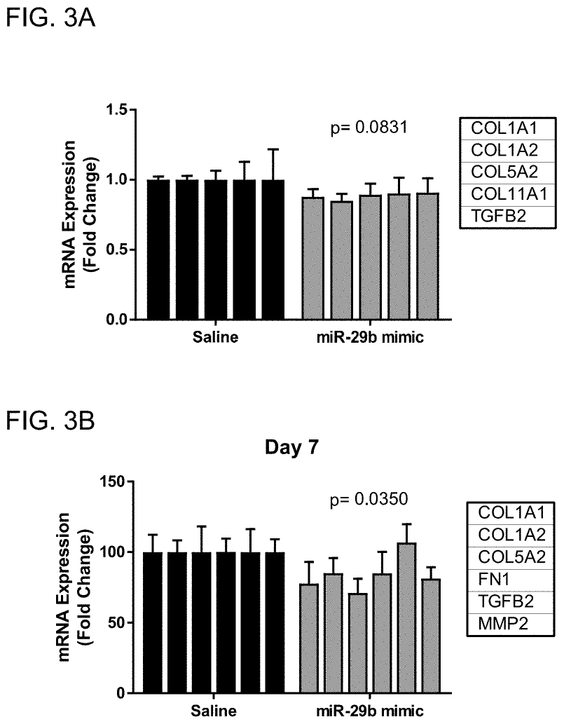

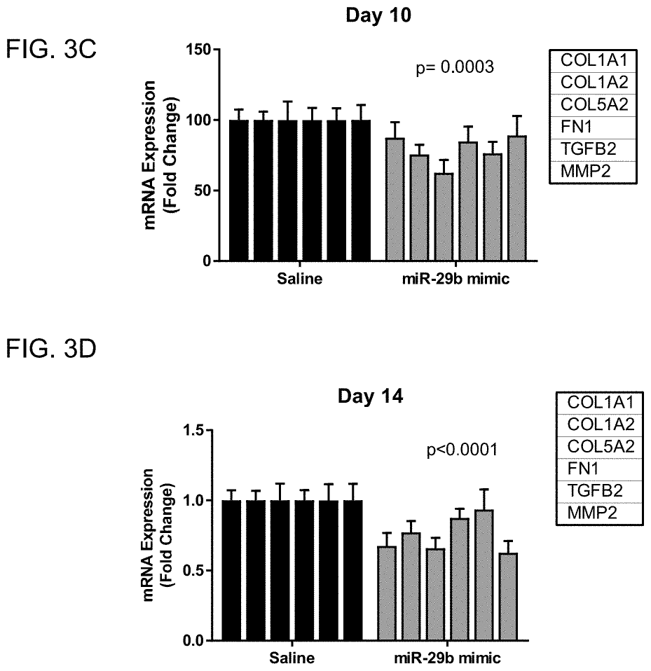

A decrease (p=0.0831) in expression was seen for multiple miR-29b target genes with miR-29b mimic treatment at day 7 (FIG. 3A). In a separate study, additional time points and genes were assessed (FIGS. 3B-3D). The three panels show data from the day 7, 10 and 14 time points respectively).

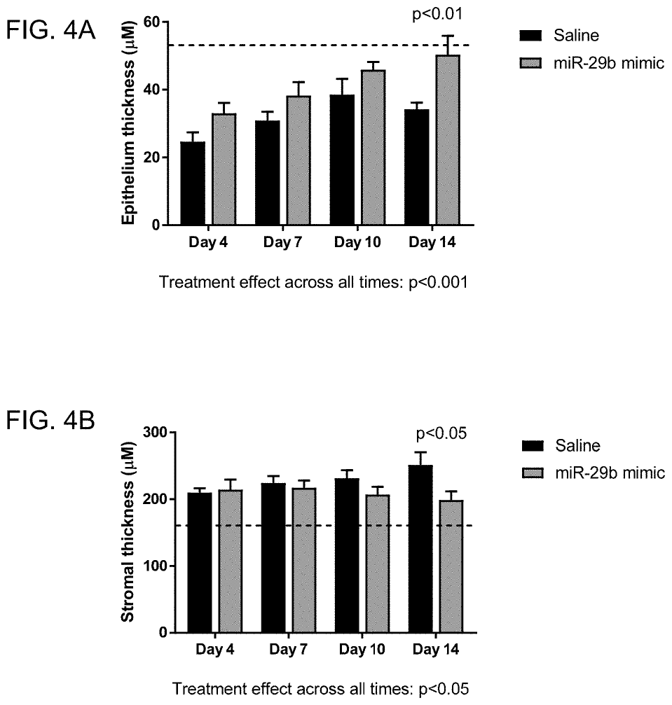

Histopathology of corneas harvested between days 4 and 14 after injury showed that the miR-29b mimic increased the epithelial thickness and decreased the stromal thickness, relative to saline treated control burned eyes (FIGS. 4A-4B). The hashed line on each of the panels represents the thickness of a normal, unburned rat cornea.

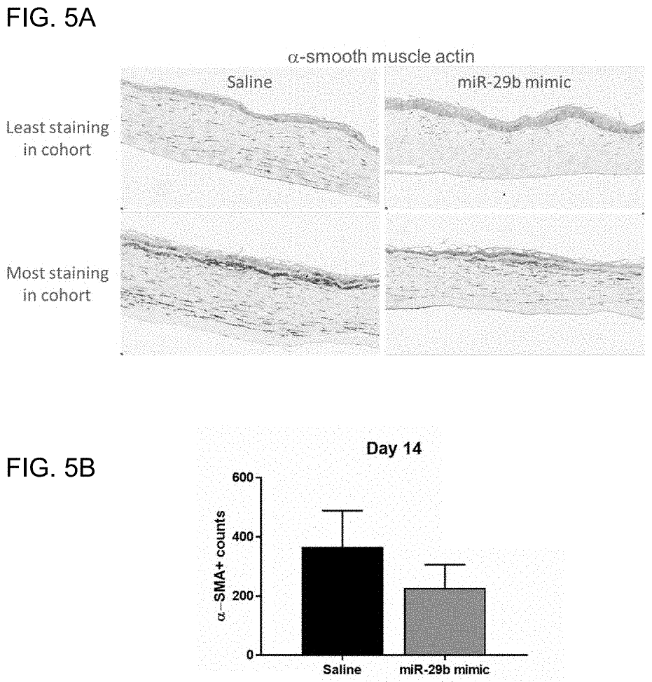

Immunohistochemistry analysis for alpha-smooth muscle actin (α-SMA) was performed on Day 14, and a decrease in a-SMA staining was observed in burned eyes treated with the miR-29b mimic, as compared to burned eyes treated with saline (FIG. 5A). Data are graphed in FIG. 5B.

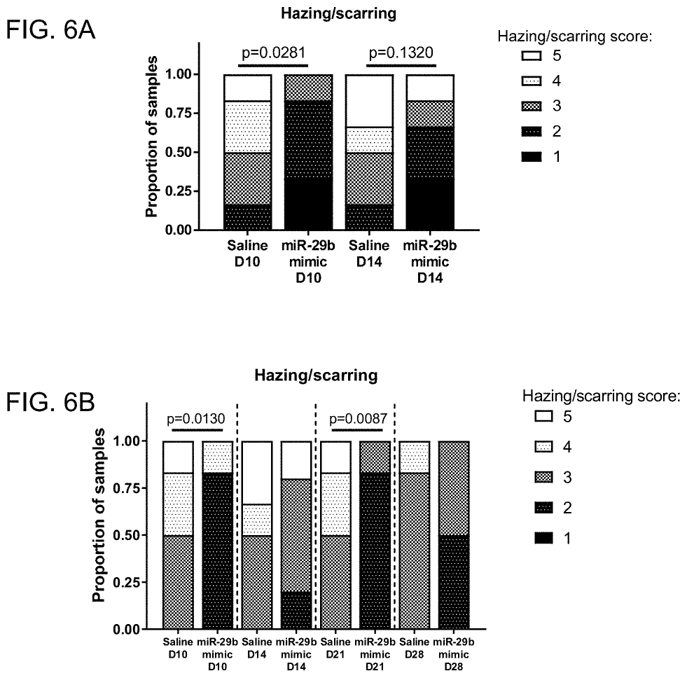

Corneal scarring and hazing were assessed clinically in two separate studies and treatment with the miR-29b mimic was found to significantly reduce the degree of corneal scarring and hazing in alkali burned eyes as compared to saline treatment in both studies (FIG. 6A and FIG. 6B).

### Example 2: Biodistribution of miR-29b mimic

Methods

Rat intravitreal administration: Rats were anesthetized with isoflurane and proparacaine and saline or miR-29b mimic was injected into the vitreous body (5 uL, 2.5 or 70 mg/mL miR-29b mimic in sterile saline). Intravitreal injection was performed once per eye.

Expression of miR-29 targets and miR-29b mimic pharmacodynamic biomarkers (COL1A1, COL1A2, COL5A2, COL11A1, MMP2, TGFB2) was assessed via qRT-PCR (reagents from Life Technologies). Pharmacokinetics was assessed by quantitative RT-PCR from whole tissue homogenates over time.

Results

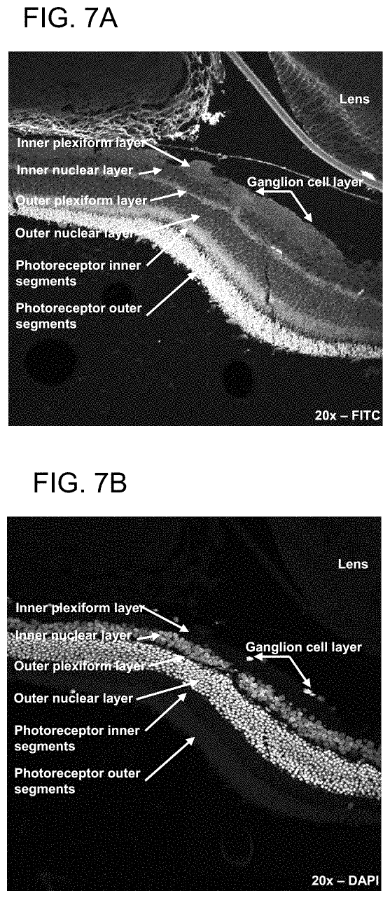

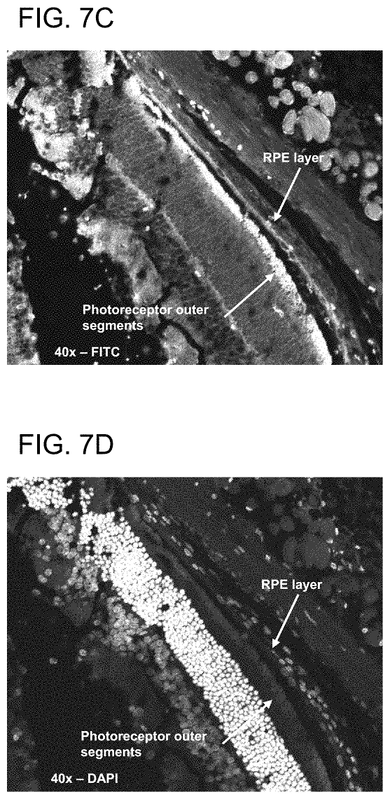

FITC-miR-29b mimic (2.5 mg/mL) was taken into all layers of the rat retina including the RPE layer after intravitreal injection (FIG. 7A and FIG. 7C). DAPI nuclear staining of the same section is shown in FIG. 7B and FIG. 7D.

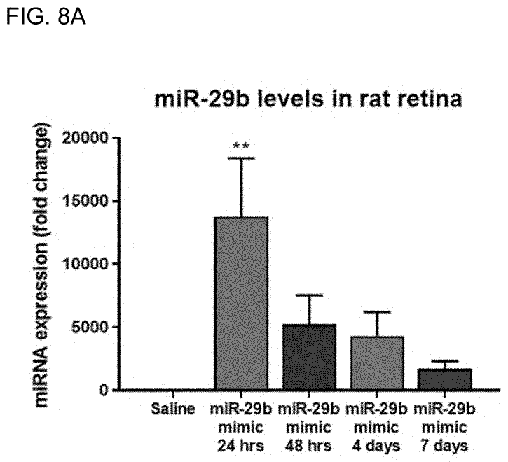

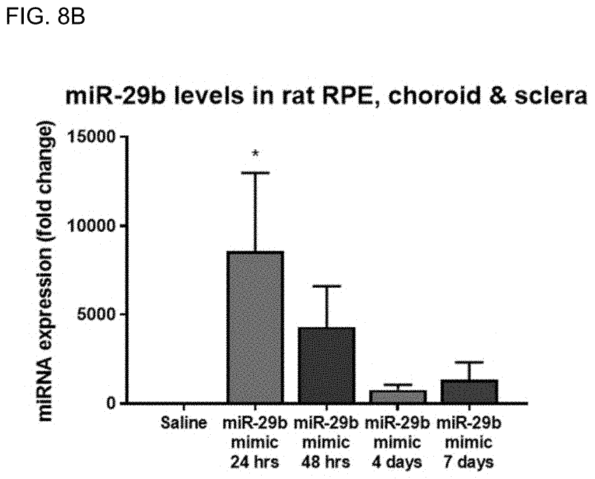

Saline or miR-29b mimic (70 mg/mL) was injected intravitreally into uninjured rat eyes. Retina and RPE/sclera/choroid were dissected from each eye between 1 and 7 days after injection application and miR-29b was quantified by qRT-PCR. miR-29b was detected at very high levels in miR-29b mimic treated eyes (13753-fold above endogenous in retina, 8595-fold above endogenous in RPE/sclera/choroid), with persistence up to the 7 day timepoint (1727-fold above endogenous in retina, 1343-fold above endogenous in RPE/sclera/choroid) (FIG. 8A and FIG. 8B).

### Example 3

Introduction of miR-29b mimic in Proliferative Vitreoretinopathy (PVR)

The pigmented New Zealand rabbit is known to have a fibrotic response following the intravitreal injection of conjunctival fibroblasts and recapitulates human proliferative vitreoretinopathy without the need to create a primary retinal detachment.

Methods

Male and female Pigmented New Zealand rabbits (1.64-2.25 kg) were anesthetized with ketamine/xylazine and 0.5% Proparacaine Hydrochloride Ophthalmic Solution was applied for local anesthesia. One eye per animal was injected once with 100 μL of approximately 5×10Λ5 Rabbit Conjunctival Fibroblasts suspended in balanced salt solution (BSS) into the vitreous body.

Rabbits were anesthetized with ketamine/xylazine and proparacaine and BSS or miR-29b mimic was injected into the vitreous body (100 uL, 10 mg/mL miR-29b mimic in BSS). Intravitreal injection was performed once weekly for one month. Uptake/distribution of a FITC labeled oligo was assessed via confocal microscopy.

miR-29 Target Expression: Total RNA was extracted from rabbit retinas treated with BSS or miR-29b mimic (10 mg/mL) using Trizol (Life Technologies). Expression of miR-29 targets and miR-29b mimic pharmacodynamic biomarkers (COL1A1, COL1A2, MMP2, TGFB2) was assessed via qRT-PCR (reagents from Life Technologies). Pharmacokinetics was assessed by quantitative RT-PCR from whole tissue homogenates over time.

Results

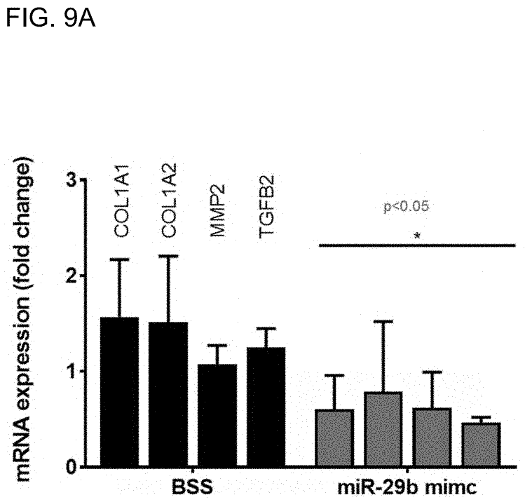

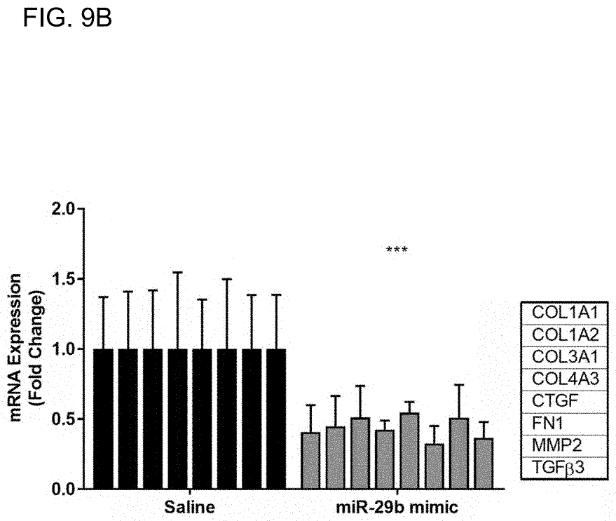

miR-29b mimic (10 mg/mL) or BSS was administered via weekly intravitreal injection into rabbit eyes that had received an allogeneic transplant of rabbit conjunctival fibroblasts (RCF), which is representative of proliferative vitreoretinopathy. Total RNA was isolated from retinas harvested at one month post-RCF injection and one week following the last injection, and expression of miR-29b target genes was quantified by qRT-PCR. miR-29b mimic significantly (p<0.05) repressed expression of multiple miR-29b target genes (FIG. 9A).

### Example 4

Introduction of miR-29b mimic in Diabetic Retinopathy

The streptozotocin-treated rat is representative of uncontrolled type 1 diabetes with prolonged hyperglycemia and has been shown to have fibrotic changes to the retina after about 10 weeks of diabetes. It recapitulates molecular and histologic changes of diabetic retinopathy without requiring the use of large animals such as non-human primates.

Methods

Diabetic retinopathy: Diabetes was induced in male Sprague Dawley rats (251-315g) by the administration of 65 mg/kg streptozotocin via IP injection. At 72 h post-injection, rats were confirmed diabetic if their fasting blood glucose levels were >300 mg/dL. Rats were given nutritional support including 0.9% saline water to drink, subcutaneous fluids, and long acting insulin as needed to maintain animal health while keeping blood glucose levels in the range of 350-650 mg/dL. Diabetic rats were maintained for a total of 11 weeks.

Rat intravitreal administration: Rats were anesthetized with isoflurane and proparacaine and saline or miR-29b mimic was injected into the vitreous body (5 uL, 2.5 or 70 mg/mL miR-29b mimic in sterile saline). Intravitreal injection was performed once per eye weekly for three weeks.

miR-29 Target Expression: Total RNA was extracted from rat retinas treated with BSS or miR-29b mimic (70 mg/mL) using Trizol (Life Technologies). Expression of miR-29 targets and miR-29b mimic pharmacodynamic biomarkers (COL1A1, COL1A2, COL3A1, COL4A3, CTGF, MMP2, TGFB2) was assessed via qRT-PCR (reagents from Life Technologies). Pharmacokinetics was assessed by quantitative RT-PCR from whole tissue homogenates over time.

Results

miR-29b mimic (70 mg/mL) or saline was administered via weekly intravitreal injection into eyes from rats that had sustained hyperglycemia, a model of diabetic retinopathy. Total RNA was isolated from retinas harvested at 11 weeks post-induction of diabetes and one week following the last injection, and expression of miR-29b target genes was quantified by qRT-PCR. miR-29b mimic significantly (p<0.001) repressed expression of multiple miR-29b target genes in this model (FIG. 9B).

### Example 5

Transfection of miR-29b mimic in Human Retinal Pigment Epithelial (RPE) Cells

Methods

In vitro Assessments: Human induced pluripotent stem cell derived retinal pigment epithelial cells (iPS-RPE, Cellular Dynamics) and primary human RPE cells (hRPE, Lonza) were grown as per the manufacturers' directions. RPE cells were treated with recombinant human TGF-β1 (TGF-β1, Peprotech) and/or actively transfected with miR-29b mimic using Dharmafect 1 transfection reagents. Total RNA was isolated at 48 hours after TGF-β1 treatment or transfection using RNeasy kits (Qiagen) or Trizol (Life Technologies), and expression of miR-29 target genes (COL1A1, COL1A2, COL3A1, FN1) was assessed via qRT-PCR (reagents from Life Technologies).

Results

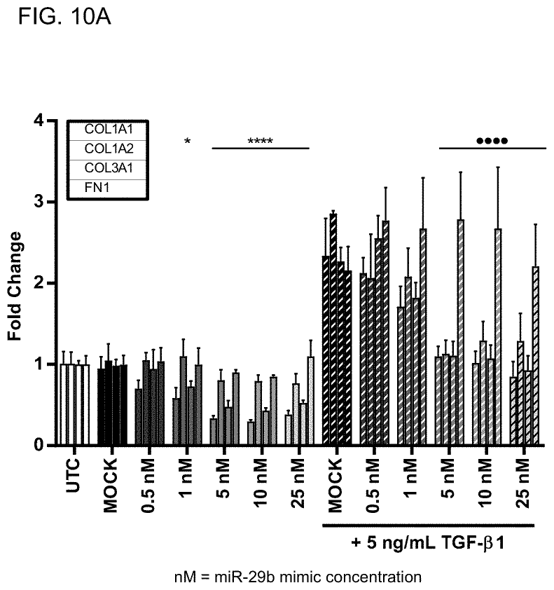

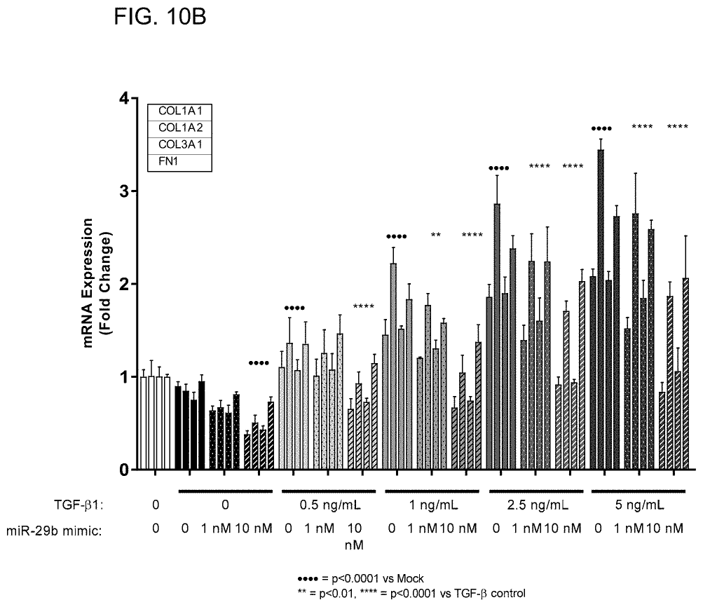

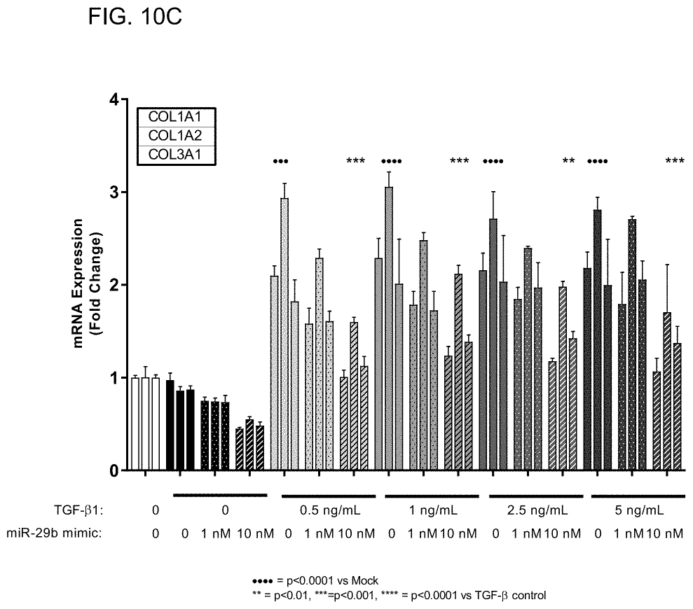

TGF-62 1 (0.5-5 ng/mL) was added and miR-29b mimic (0.5-25 nM) was actively transfected into iPS-RPE cells (FIG. 10A and FIG. 10B) or hRPE cells (FIG. 10C). Total RNA was extracted at 48 hours after treatment and expression of miR-29b target genes was quantified by qRT-PCR. In both cell lines, TGF-β1 treatment significantly upregulated expression of multiple miR-29b target genes. Additionally, miR-29b mimic repressed expression of multiple miR-29b target genes in the presence and absence of TGF-β1 treatment.

1. A method of treating ocular fibrosis in a subject comprising administering to the subject a therapeutically effective amount of a miR-29 mimetic compound, wherein the miR-29 mimetic compound comprises:
(a) a first strand of 23 to 26 ribonucleotides comprising a mature miR-29a, miR-29b, or miR-29c sequence; and
(b) a second strand of 22 to 23 ribonucleotides comprising a sequence that is substantially complementary to the first strand and having at least one modified nucleotide, wherein the first strand has a 3′ nucleotide overhang relative to the second strand,
wherein the ocular fibrosis comprises retinal fibrosis, corneal fibrosis, conjunctival fibrosis, or fibrosis of the trabecular meshwork.

2. The method of claim 1, wherein the ocular fibrosis results from an ocular disease or disorder selected from the group consisting of diabetic retinopathy, bacterial infection, viral infection, fungal infection, amoeba infection, ocular trauma, chemical corneal burn, thermal corneal burn, pterygium, glaucoma, surgical trauma, Fuch's Endothelial Corneal Dystrophy (FECD), and anterior proliferative vitreoretinopathy (anterior PVR).

3. A method of treating an ocular disease or disorder in a subject comprising administering to the subject a therapeutically effective amount of a miR-29 mimetic compound, wherein the miR-29 mimetic compound comprises:
(a) a first strand of 23 to 26 ribonucleotides comprising a mature miR-29a, miR-29b, or miR-29c sequence; and
(b) a second strand of 22 to 23 ribonucleotides comprising a sequence that is substantially complementary to the first strand and having at least one modified nucleotide, wherein the first strand has a 3′ nucleotide overhang relative to the second strand,
wherein the ocular disease or disorder is selected from the group consisting of diabetic retinopathy, bacterial infection, viral infection, fungal infection, amoeba infection, ocular trauma, chemical corneal burn, thermal corneal burn, pterygium, glaucoma, surgical trauma, Fuch's Endothelial Corneal Dystrophy (FECD), and anterior proliferative vitreoretinopathy (anterior PVR).

4. The method of claim 3, wherein the ocular disease or disorder comprises an ocular fibrosis selected from the group consisting of comprises retinal fibrosis, corneal fibrosis, conjunctival fibrosis, or fibrosis of the trabecular meshwork.

5. The method of claim 1, wherein the miR-29 mimetic compound is administered via iontophoresis, subconjunctival injection, injection into the pterygium, injection at site of a trabecular bleb, sub-tendon injection, an intravitreal injection, intracamerally, or topically.

6. The method of claim 1, wherein the ocular fibrosis comprises proliferative vitreoretinopathy.

7. The method of claim 1, wherein the ocular fibrosis comprises retinal fibrosis associated with diabetic retinopathy.

8. The method of claim 1, wherein the ocular fibrosis comprises retinal fibrosis associated with anti-VEGF treatment.

9. The method of claim 1, wherein the ocular fibrosis comprises corneal hazing or corneal scarring.

10. The method of claim 1, wherein the ocular fibrosis results from a corneal burn.

11. The method of claim 1, wherein the ocular fibrosis results from pterygium.

12. The method of claim 1, wherein the ocular fibrosis results from Fuch's Endothelial Corneal Dystrophy (FECD).

13. The method of claim 1, wherein the ocular fibrosis results from glaucoma.

14. The method of claim 1, wherein the ocular fibrosis comprises a trabecular bleb.

15. The method of claim 1, wherein the miR-29 mimetic compound is administered topically into the cornea.

16. The method of claim 15, wherein the miR-29 mimetic compound is administered topically into the cornea in a drop formulation.

17. The method of claim 15, wherein the miR-29 mimetic compound is administered topically into the cornea once daily, twice daily, three times daily, or four times daily.

18. The method of claim 15, wherein the miR-29 mimetic compound is administered topically periodically for up to one week, two weeks, three weeks, four weeks, or 28 days.

19. The method of claim 1, wherein the miR-29 mimetic compound is administered in a drop formulation.

20. The method of claim 1, wherein the miR-29 mimetic compound is administered via intravitreal injection once weekly.

21. The method of claim 1, wherein the miR-29 mimetic compound is administered via intravitreal injection periodically for one month.

22. The method of claim 1, wherein the expression of COL1A1, COL1A2, COL3A1, COL4A3, COL5A2, COL11A1, FN1, MMP2, CTGF, TGFB2, and/or TGFB3 is reduced.

23. The method of claim 1, wherein the miR-29 mimetic compound is presented in Table 2.

24. The method of claim 1, wherein the first strand comprises a sequence selected from SEQ ID NO: 2, 18-21, or 31-34 and the second strand comprises a sequence selected from 1, 8-10, 13-17, or 28-30.

25. The method of claim 1 , wherein the miR-29 mimetic compound comprises:
(a) a first strand of about 23 to about 26 ribonucleotides comprising a mature miR-29b sequence, wherein the first strand comprises a sequence selected from SEQ ID NO: 2, 18-21, and 31-34; and
(b) a second strand of about 22 to about 25 ribonucleotides comprising a sequence that is substantially complementary to the first strand and having at least one modified nucleotide, wherein the first strand has a 3′ nucleotide overhang relative to the second strand.

26. The method of claim 1, wherein the miR-29 mimetic compound comprises:
(a) a first strand of about 23 to about 26 ribonucleotides comprising a mature miR-29b sequence; and
(b) a second strand of about 22 to about 25 ribonucleotides comprising a sequence that is substantially complementary to the first strand and having at least one modified nucleotide, wherein the first strand has a 3′ nucleotide overhang relative to the second strand, and wherein the second strand comprises a sequence selected from SEQ ID NO: 1, 8-10, 13-17, and 28-30.

27. The method of claim 1, wherein the first strand comprises the sequence of SEQ ID NO: 19 and the second strand comprises the sequence of SEQ ID NO: 1.

28. The method of claim 1, wherein the first strand comprises the sequence of SEQ ID NO: 19 and the second strand comprises the sequence of SEQ ID NO: 15.

29. The method of claim 1, wherein the first strand comprises a mature miR-29a sequence.

30. The method of claim 1, wherein the miR-29 mimetic compound is presented in Table 1.

31. The method of claim 1, wherein the first strand comprises a sequence of SEQ ID NO: 6, 7 or 27 and the second strand comprises a sequence selected from SEQ ID NO: 3-5 or 11.

32. The method of claim 1, wherein the first strand comprises a mature miR-29c sequence.

33. The method of claim 1 , wherein the miR-29 mimetic compound is presented in Table 3.

34. The method of claim 1, wherein the first strand comprises a sequence of SEQ ID NO: 25, 26, or 35 and the second strand comprises a sequence selected from SEQ ID NO: 22-24 or 12.

35. The method of claim 1 , wherein a therapeutically effective amount of the miR-29 mimetic compound is comprised in a pharmaceutical composition.

36. The method of claim 1, wherein the subject is a human.

37. The method of claim 36, wherein the subject suffers from diabetes.

38. The method of claim 36, wherein the subject suffers from diabetic retinopathy.

39. The method of claim 36, wherein the subject suffers from age-related macular degeneration (AMD).

40. The method of claim 39, wherein the subject suffers from wet age-related macular degeneration (wet AMD).

41. The method of claim 38, wherein the subject is receiving an anti-VEGF therapy.
